SEQUENCE LISTING (57 sequences)

SEQ ID NO: 1 - DNA, 6507 bp, Artificial Sequence
  Synthetic plasmid
  misc_feature: plasmid with a sequence from Indiana vesiculovirus
gagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatccctcgaggaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcctcgagggatccgtcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacggatcggccgcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctaga

SEQ ID NO: 2 - protein, 511 aa, Alagoas vesiculovirus
MTPAFILCMLLAGSSWAKFTIVFPQSQKGDWKDVPPNYRYCPSSADQNWHGDLLGVNIRAKMPKVHKAIKADGWMCHAAKWVTTCDYRWYGPQYITHSIHSFIPTKAQCEESIKQTKEGVWINPGFPPKNCGYASVSDAESIIVQATAHSVMIDEYSGDWLDSQFPTGRCTGSTCETIHNSTLWYADYQVTGLCDSALVSTEVTFYSEDGLMTSIGRQNTGYRSNYFPYEKGAAACRMKYCTHEGIRLPSGVWFEMVDKELLESVQMPECPAGLTISAPTQTSVDVSLILDVERMLDYSLCQETWSKVHSGLPISPVDLGYIAPKNPGAGPAFTIVNGTLKYFDTRYLRIDIEGPVLKKMTGKVSGTPTKRELWTEWFPYDDVEIGPNGVLKTPEGYKFPLYMIGHGLLDSDLQKTSQAEVFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 3 - DNA, 1536 bp, Alagoas vesiculovirus
atgactcccgcatttatcttgtgcatgctcttggcaggcagttcttgggcaaaatttactattgtctttcctcaaagtcaaaagggagactggaaagatgtccctccaaattatagatattgtccatctagcgcagaccaaaactggcatggagacttgttaggagttaatatcagagcaaagatgccaaaagtgcataaggcaatcaaggctgatggctggatgtgtcatgctgccaagtgggtcacaacatgtgattatagatggtatgggcctcaatacatcacgcactccatccactccttcatccctactaaagctcagtgtgaggaaagcataaagcagactaaggaaggagtttggatcaatccaggatttcccccaaagaactgcggatatgcttcagtaagtgatgctgaatcaattatagtccaagccactgcccactctgtgatgattgatgaatactcaggagactggcttgactctcaattcccaactggtagatgcacgggctccacctgcgaaacaatccacaattctacattgtggtatgccgattatcaagtgaccggcctgtgcgactctgctcttgtctcgacagaagtcactttttactcagaagatggtctaatgacatcaatagggagacagaacacaggttatcgaagtaactacttcccctatgagaaaggagcagctgcatgtcgaatgaagtactgtacacatgaaggaatccgactgccctcaggtgtgtggtttgaaatggttgacaaggagctgctggagtctgttcaaatgccagaatgcccagctggcctaaccatttcagccccgactcagacctctgttgatgtgagcttgattttggatgtggagcggatgttggactattcattgtgtcaggagacgtggagcaaggttcatagcggattgccaatatctcccgtggatcttggatatatagctccaaaaaacccaggtgctggtcctgctttcacaattgtcaatgggactcttaaatacttcgacacaagatacttgagaattgacatcgagggaccagtccttaagaagatgacaggcaaagtcagtggcaccccgactaagcgtgagttgtggactgagtggtttccctatgatgatgtggaaatcggacctaacggagttcttaaaactcctgaaggatacaaatttcctctctacatgatcggacacgggctgctggactcagatcttcaaaagacatcgcaagctgaggtgttccaccatccgcagattgctgaagcagtccaaaagctaccagatgatgagacacttttctttggagacaccgggatttcaaaaaaccccgtggaagtcattgaggggtggttcagcaactggcgcagttctgtcatggcaatagtgttcgccatcttgctgcttgtgatcacagtcttgatggtccgcttatgtgtagcatttcgacatttctgctgccaaaaaagacacaaaatatacaatgatttggaaatgaatcaactacggagataa

SEQ ID NO: 4 - protein, 517 aa, Arizona vesiculovirus
MLSYLILAIIVSPILGKIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYNDGVSFNPGFPPQSCGYGTVTDAEAHIITVTPHSVKVDEYTGEWIDPHFIGGRCKGKICETVHNSTKWFTSSDGESVCSQLFTLVGGTFFSDSEEITSMGLPETGMRSNYFPYISTEGICKMPFCRKPGYKLKNDLWFQITDPDLDKTVRDLPHIKDCDLSSSIITPGEHATDISLISDVERILDYALCQNTWSKIEAGEPITPVDLSYLGPKNPGVGPVFTVINGSLHYFTSKYLRVELESPVIPRMEGRVAGTKIVRQLWDQWFPFGEAEIGPNGVLKTKQGYKFPLHIIGTGEVDSDIKMERIVKHWEHPHIEAAQTFLKKDDTEEVIYYGDTGVSKNPVELVEGWFSGWRSSIMGVVAVIIGFVILIFLIRLIGVLSSLFRQKRRPIYKSDVEMTHFR

SEQ ID NO: 5 - DNA, 1668 bp, Arizona vesiculovirus
atgttcatgccttcttctctttcctacagctcctgggcaacgtgctggttgttgtgctgtctcatcattttggcaaagaattcctcgacggatccctcgaggaattctgacactatgttgtcttatctaattcttgcaattattgtttcgcctattttaggcaaaattgaaatcgtcttccctcagcatactactggagattggaagagggttcctcatgaatacaattactgtcccactagtgcagataaaaactcacatgggactcagacaggaattcctgttgagctaacaatgcccaagggactaacaacacatcaggttgatgggtttatgtgtcactctgctttatggatgaccacttgtgatttcagatggtatggacctaaatacataacccactctatacataatgaggagcctacagattaccaatgtttggaagccatcaaggcatataacgatggtgttagctttaatccagggttccctcctcagagctgtgggtatggtacggtcacggacgctgaagcccatattataacagtcactcctcactctgttaaagtagatgagtacactggagagtggattgacccacatttcatcggggggagatgcaagggcaaaatttgtgaaacagtccacaactccacaaaatggtttacatcttcagatggagaaagtgtctgtagtcaattattcactctagttggaggaacttttttctctgactcagaggaaattacttcaatgggactaccagaaacagggatgaggagtaattattttccttacatatccacagagggaatatgcaagatgccgttctgcagaaagccagggtacaaacttaagaatgacctctggtttcagatcacggatccagatttggataaaacagttagagatcttccgcacatcaaagattgtgatctctcctcatccattataacaccaggggaacatgcaacagacatatccctgatatcagatgtggaaagaatcctggattatgctctttgtcaaaacacatggagcaaaattgaagccggagaaccaatcactcctgtagatctcagctaccttggaccaaagaatcccggagtaggcccggtttttaccgtcataaatggttctttgcattacttcacatcaaaatatctgcgtgtggaactggaaagtcctgttatacccagaatggaagggagagttgcaggaactaaaattgtgcggcaattgtgggatcaatggttcccttttggagaggctgagattggacccaatggtgtgttgaagaccaagcaaggatacaaattcccattacacatcattggaacaggagaggtagacagtgacatcaaaatggagaggattgttaaacactgggaacacccccacattgaagccgctcagacatttttaaaaaaagatgatacagaagaagtcatctattatggcgacacaggggtatcaaaaaacccagttgagttagttgagggctggtttagtggatggaggagctctatcatgggagtggtggctgtgattatcggattcgtgattttaatatttttaattagactgattggagtcctatccagtctttttagacaaaaaagaaggccaatttataaatcggatgtagagatgacccacttccgttaa

SEQ ID NO: 6 - protein, 523 aa, Carajas vesiculovirus
MKMKMVIAGLILCIGILPAIGKITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 7 - DNA, 1572 bp, Carajas vesiculovirus
atgaagatgaaaatggtcatagcaggattaatcctttgtatagggattttaccggctattgggaaaataacaatttctttcccacaaagcttgaaaggagattggaggcctgtacctaagggatacaattattgtcctacaagtgcggataaaaatctccatggtgatttgattgacataggtctcagacttcgggcccctaagagcttcaaagggatctccgcagatggatggatgtgccatgcggcaagatggatcaccacctgtgatttcagatggtatggacccaagtacatcacccactcaattcactctttcaggccgagcaatgaccaatgcaaagaagcaatccggctgactaatgaagggaattggattaatccaggtttccctccgcaatcttgcggatatgcttctgtaaccgactcagaatccgttgtcgtaaccgtgaccaagcaccaggtcctagtagatgagtactccggctcatggatcgatagtcaattccccggaggaagttgcacatcccccatttgcgatacagtgcacaactcgacactttggcacgcggaccacaccctggacagtatctgtgaccaagaattcgtggcaatggacgcagttctgttcacagagagtggcaaatttgaagagttcggaaaaccgaactccggcatcaggagcaactattttccttatgagagtctgaaagatgtatgtcagatggatttctgcaagaggaaaggattcaagctcccatccggtgtctggtttgaaatcgaggatgcagagaaatctcacaaggcccaggttgaattgaaaataaaacggtgccctcatggagcagtaatctcagctcctaatcagaatgcagcagatatcaatctgatcatggatgtggaacgaattctagactactccctttgccaagcaacttggagcaaaatccaaaacaaggaagcgttgacccccatcgatatcagttatcttggtccgaaaaacccaggaccaggcccagccttcaccataataaatggaacactgcactacttcaatactagatacattcgagtggatattgcagggcctgttaccaaagagattacaggatttgtttcgggaacatctacatctagggtgctgtgggatcagtggttcccatatggagagaattccattggacccaatggcttgctgaaaaccgccagcggatacaaatatccattgttcatggttggtacaggtgtgctggatgcggacatccacaagctgggagaagcaaccgtgattgaacatccacatgccaaagaggctcagaaggtagttgatgacagtgaggttatattttttggtgacaccggagtctccaagaatccagtggaggtagtcgaaggatggtttagcggatggagaagctctttgatgagcatatttggcataattttgttgattgtttgtttagtcttgattgttcgaatccttatagcccttaaatactgttgtgttagacacaaaaagagaactatttacaaagaggaccttgaaatgggtcgaattcctcggagggcttaa

SEQ ID NO: 8 - protein, 511 aa, Indiana vesiculovirus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 9 - DNA, 1536 bp, Indiana vesiculovirus
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 10 - protein, 512 aa, Maraba vesiculovirus
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNYFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 11 - DNA, 1539 bp, Maraba vesiculovirus
atgttgagactttttctcttttgtttcttggccttaggagcccactccaaatttactatagtattccctcatcatcaaaaagggaattggaagaatgtgccttccacatatcattattgcccttctagttctgaccagaattggcataatgatttgactggagttagtcttcatgtgaaaattcccaaaagtcacaaagctatacaagcagatggctggatgtgccacgctgctaaatgggtgactacttgtgacttcagatggtacggacccaaatacatcacgcattccatacactctatgtcacccaccctagaacagtgcaagaccagtattgagcagacaaagcaaggagtttggattaatccaggctttccccctcaaagctgcggatatgctacagtgacggatgcagaggtggttgttgtacaagcaacacctcatcatgtgttggttgatgagtacacaggagaatggattgactcacaattggtggggggcaaatgttccaaggaggtttgtcaaacggttcacaactcgaccgtgtggcatgctgattacaagattacagggctgtgcgagtcaaatctggcatcagtggatatcaccttcttctctgaggatggtcaaaagacgtctttgggaaaaccgaacactggattcaggagtaattactttgcttacgaaagtggagagaaggcatgccgtatgcagtactgcacacaatgggggatccgactaccttctggagtatggtttgaattagtggacaaagatctcttccaggcggcaaaattgcctgaatgtcctagaggatccagtatctcagctccttctcagacttctgtggatgttagtttgatacaagacgtagagaggatcttagattactctctatgccaggagacgtggagtaagatacgagccaagcttcctgtatctccagtagatctgagttatctcgccccaaaaaatccagggagcggaccggccttcactatcattaatggcactttgaaatatttcgaaacaagatacatcagagttgacataagtaatcccatcatccctcacatggtgggaacaatgagtggaaccacgactgagcgtgaattgtggaatgattggtatccatatgaagacgtagagattggtccaaatggggtgttgaaaactcccactggtttcaagtttccgctgtacatgattgggcacggaatgttggattccgatctccacaaatcctcccaggctcaagtcttcgaacatccacacgcaaaggacgctgcatcacagcttcctgatgatgagactttattttttggtgacacaggactatcaaaaaacccagtagagttagtagaaggctggttcagtagctggaagagcacattggcatcgttctttctgattataggcttgggggttgcattaatcttcatcattcgaattattgttgcgattcgctataaatacaaggggaggaagacccaaaaaatttacaatgatgtcgagatgagtcgattgggaaataaataa

SEQ ID NO: 12 - protein, 513 aa, Morreton vesiculovirus
MLVLYLLLSLLALGAQCKFTIVFPHNQKGNWKNVPANYQYCPSSSDLNWHNGLIGTSLQVKMPKSHKAIQADGWMCHAAKWVTTCDFRWYGPKYVTHSIKSMIPTVDQCKESIAQTKQGTWLNPGFPPQSCGYASVTDAEAVIVKATPHQVLVDEYTGEWVDSQFPTGKCNKDICPTVHNSTTWHSDYKVTGLCDANLISMDITFFSEDGKLTSLGKEGTGFRSNYFAYENGDKACRMQYCKHWGVRLPSGVWFEMADKDIYNDAKFPDCPEGSSIAAPSQTSVDVSLIQDVERILDYSLCQETWSKIRAHLPISPVDLSYLSPKNPGTGPAFTIINGTLKYFETRYIRVDIAGPIIPQMRGVISGTTTERELWTDWYPYEDVEIGPNGVLKTATGYKFPLYMIGHGMLDSDLHISSKAQVFEHPHIQDAASQLPDDETLFFGDTGLSKNPIELVEGWFSGWKSTIASFFFIIGLVIGLYLVLRIGIALCIKCRVQEKRPKIYTDVEMNRLDR

SEQ ID NO: 13 - DNA, 1542 bp, Morreton vesiculovirus
atgctggttttatacctgttattgagccttttggctctgggagctcaatgcaagttcactatagtatttcctcacaatcaaaaagggaattggaaaaatgtaccggcaaattatcagtattgtccttctagttctgacttgaattggcacaatgggctgattggcacttctctccaagtcaaaatgcccaaaagccataaggccatccaagcggatggttggatgtgtcatgctgccaagtgggtgactacttgtgacttcagatggtacggacctaaatatgtgacacattctataaagtccatgatacctacagtcgaccagtgtaaagaaagtatagcccagactaaacaaggaacgtggttaaatccgggtttccctccccaaagttgtggatatgcttccgttacagatgcagaggctgtgatagtcaaagcaaccccccaccaggttttggttgacgaatatacaggagaatgggttgactcccaatttccgactggaaaatgcaataaagacatttgcccaacagttcacaactcaactacctggcactcagattataaggtcactggcctttgcgatgcaaatttgatctcaatggacatcactttcttctccgaagatggaaaattaacatccctcgggaaagaaggaacagggttcagaagcaattactttgcatacgaaaatggtgacaaagcatgccgcatgcagtactgtaaacactggggagttcgacttccatccggagtgtggttcgaaatggcagataaagacatctataatgatgcgaaattcccggattgccctgaaggatcatccattgcggctccctctcagacttcagtcgatgttagtctcattcaggatgtagagagaatcttggactactctttgtgtcaggaaacctggagcaaaattcgtgctcatttgcccatttcaccagttgacctcagctatttatccccaaaaaatcctggaactggtcctgcattcactatcatcaatgggacattaaaatactttgagactcgatacataagagtcgatatcgcaggacccatcattcctcaaatgagaggagtaatcagcggaaccacgaccgagagagagctgtggacggactggtacccctacgaagatgttgaaatcggaccaaatggggttttgaaaactgctacagggtataagttccctttatacatgattgggcacggcatgctcgactcagatctccacatctcatcaaaggctcaggtttttgaacatccccatattcaggatgctgcttctcagcttcctgatgatgagactttattttttggtgatactggactctcgaaaaaccccatagagcttgtagaaggttggttcagcggatggaaaagcactattgcttcttttttcttcataatagggcttgtgatcggattatatttggttcttaggattggaatcgctttatgcatcaaatgccgagtgcaggagaaaaggcccaaaatttacactgatgtggaaatgaacagattggatcgatga

SEQ ID NO: 14 - protein, 517 aa, New Jersey vesiculovirus
MLSYLILALTISPILGKIEIVFPQHTTGDWKRVPHEYNYCPTSADKNSHGTQTGIPIELTMPKGLTTHQVEGFMCHAALWVTTCDFRWYGPKYITHSIHNEEPTDYQCLEAIKAYKDGASFNPGFPPQSCGYGSVTDAEAHIITITPHSVKVDEYTGEWIDPHFIGGRCKGKTCETVHNSTKWFTSSDGESVCSQLFTLVRGTFFSDSEEITSIGLPETGIRSNYFPYVSTEGICKMPFCRKPGYKLKNDLWFQIADPDLDQKVKDLPHIKDCDLSSSIITPGEHATDISLISDVERILDYALCQNTWSKIEAGEPITPVDISYLGPKNPGVGPVFTIINGSLHYFTSKYLRVELENPVIPRMEGKVAGTRIVRQLWDQWFPFGEAEIGPNGVLKTKQGYKFPLHIVGTGEVDNDIKMERIVKHWEHPHIEAAQTFLKKDDTEEVIYYGDTGVSKNPVELVEGWFSGWRSSIMGVLAVIIGFVILIFLIRLIGLMSNFCKPRRGPIYKSDVEMAHFR

SEQ ID NO: 15 - DNA, 1554 bp, New Jersey vesiculovirus
atgttgtcttacctcatccttgcacttaccatctcgcccatactgggcaaaattgaaattgtctttccccaacataccacaggggattggaaaagagtgccacatgagtacaattattgccctaccagtgcggacaaaaactcccacggaactcaaacagggattcctattgagttgacaatgcctaaaggactaacaacccatcaagtagagggatttatgtgtcatgcagctttatgggtgaccacttgtgattttagatggtatggaccaaaatatataactcattccatacataatgaggaaccgacagactatcagtgcctggaggccattaaagcatataaagatggagctagcttcaatcctgggtttcctcctcaaagctgtggatatggttcagtgacggatgcagaggcacacataatcacaattactcctcattctgttaaagtggatgagtatactggagaatggattgaccctcattttataggaggaaggtgcaaagggaaaacctgtgaaacagttcataattcaaccaagtggtttacatcttcagatggggaaagtgtatgcagccaattattcaccttggttagaggaacttttttctctgactcagaggagattacctcaataggattaccagaaaccggaatcaggagcaattacttcccctatgtgtctacagaaggaatttgcaaaatgccattctgcagaaagccagggtataagcttaagaatgacctctggttccaaattgcagacccagacttggatcaaaaagtgaaggatctaccacacataaaagactgtgacctttcttcctctatcatcaccccaggggaacatgcaacagacatatctctaatatcagatgtggaacggatacttgattatgctctttgtcaaaatacctggagcaagattgaagcaggagaaccaattactcctgtagacatcagctatctaggacctaaaaaccctggcgttgggccggttttcacaatcatcaacggctcactacattactttacctccaagtatctacgagttgagttagaaaatcctgttatacccagaatggaagggaaagttgcggggacccgaattgttcgtcaactgtgggaccaatggttcccttttggagaggcagagattggacctaatggtgttctgaaaaccaagcaaggatataaattcccattgcacatcgttgggactggggaagtcgacaatgacatcaaaatggaaaggattgtaaagcattgggagcacccacacatagaagctgctcagacgttcttaaaaaaagatgacacagaggaggtaatctattatggggacactggagtatccaaaaatcccgttgaattagtagagggatggttcagcggttggagaagctcaatcatgggagtgttggctgtgatcataggttttgtaatcttaatatttttaattagattgattggactgatgtccaatttctgtaaaccaagaagagggccaatctacaaatccgacgtagaaatggctcacttccggtaa

SEQ ID NO: 16 - protein, 629 aa, Bas-congo virus
MTRLSHAITKLLLLFCLTAIHAIVINYPTACHTYQEVLYQGLECPEPAISYKLDNNETVAYGQICRPQLASKDILEGYLCYKDTYISSCEETWYFTSQVKQTIVHEHVSDAECIESLAYYKSGIVETPMFLNVDCYWNAINSIKKSYLIIVYHPVPFDPYTNSIKDAVVKNSEDVNSWIRDTHYPFTKWIRDFNGTAEEKCDAQHWECFKVNLYKGWIYSPPHTKNTIGSSTQTGLILESDIYSHTLIRDLCRFQFCGIHGFVFQDQSWWDLQLNVSLSSLISTEHLSGAPDGHCKKVNEIGHAELEPNWEKILSVDDYDIRHQLCLDTLASVLGGGFLTARDLLKFAPMRPGLGPAYFLFNPNKRERAVHVWTAGATTSSILWKSTCKYELIDIPQLNDTGIITYEKLDNIIGKILRNDVGVSFKDLGFTENELTDDDVSQSQLNSSLGIYHRNTSMKGIPWKRHRASTPKLKMGPNGILHDLNAKIIHLPQASSSVFKLPPHLYEGHRVVFFNHITKKKIYEDLSKREGNDPYNVDIGDLIGRHLNRTTIPDQLHDWVSGIKRHIFSVFEQFGSLIKVVVFIIMLVLCIKIINLIYRFYKVRKSNHKKLASRKEKLHLSDPFSVNSK

SEQ ID NO: 17 - DNA, 1890 bp, Bas-congo virus
atgacccgcctgtcccacgccatcacaaaacttcttctgctcttttgtctcactgcaatacacgctattgtaatcaattacccaacagcttgccatacatatcaagaagttctttaccaaggattagaatgtcctgaacctgcaatatcctacaagttggataacaatgagacagttgcttatgggcaaatttgcagaccacagttagcatcaaaggacatattagaaggttatctctgttacaaagacacttacatatcatcttgtgaagaaacatggtatttcacatcccaggtaaagcagacaatagttcatgaacatgttagcgatgctgaatgcattgaatccttggcttactacaaaagtggtattgttgaaacccccatgtttctaaatgtagactgctattggaacgcaataaatagtatcaaaaagtcgtacttgattattgtatatcatcctgttccatttgatccctacaccaattctattaaagatgcagtggtcaaaaactcggaagatgttaactcatggatacgagacactcattacccctttactaaatggattagagattttaatggtacagctgaagaaaaatgtgacgctcagcattgggagtgtttcaaggtcaatctatataaaggttggatatactctcccccacatactaagaacaccattggctcatctacccaaactggactcatcctcgaaagtgacatctactcacacactctgattagagatctatgcagattccaattttgtggaattcacgggtttgttttccaggatcaatcatggtgggatcttcaactcaatgtgtctttatcatctttaatctctactgaacatctctccggagctcctgatggtcattgcaaaaaagtgaacgaaataggccatgctgaattagaaccgaattgggaaaagatattatcagtggatgactatgacatcaggcatcagctctgtctagacacattagcatctgttttgggaggaggctttttgacggcgcgagacctgttaaaatttgctcccatgagaccaggattaggtccagcttactttctattcaaccccaataagagagaaagagccgtgcatgtttggacagcaggggccaccacatcttccatactctggaaaagtacatgtaaatacgaacttattgatattcctcaactgaacgacacaggaataatcacttatgaaaaattagataacatcattgggaaaatcctcagaaatgatgtgggagtttcattcaaggatcttggattcaccgaaaatgagctaacagatgatgatgtctctcagtctcagcttaattcttcacttggcatttatcatagaaacacatcaatgaaggggataccatggaaaaggcatagagcatcaactcctaaattgaagatggggcctaatgggatattacatgatttgaatgcaaagattatacaccttccacaagcttcttcttctgtattcaagttacccccacacctgtatgaaggacacagggtggtgttttttaatcatataacaaagaagaagatatacgaagatttatcaaaaagagaagggaacgacccatataatgtcgacataggtgacctgatcggaaggcatctaaatagaacaacaataccagaccagttgcacgactgggtgtctgggatcaaaagacacatcttttctgtttttgaacaattcggtagtctgatcaaagttgttgtctttataataatgctagtgttgtgtataaaaatcattaatctgatatatcggttttacaaggtgaggaaatctaatcacaaaaaactagcttcacggaaagagaaacttcacctatcagatccgttctctgtaaattctaaatga

SEQ ID NO: 18 - protein, 530 aa, Chandipura virus
MTSSVTISVILLISFIAPSYSSLSIAFPENTKLDWKPVTKNTRYCPMGGEWFLEPGLQEESFLSSTPIGATPSKSDGFLCHAAKWVTTCDFRWYGPKYITHSIHNIKPTRSDCDTALASYKSGTLVSPGFPPESCGYASVTDSEFLVIMITPHHVGVDDYRGHWVDPLFVGGECDQSYCDTIHNSSVWIPADQTKKNICGQSFTPLTVTVAYDKTKEIAAGAIVFKSKYHSHMEGARTCRLSYCGRNGIKFPNGEWVSLDVKTKIQEKPLLPLFKECPAGTEVRSTLQSDGAQVLTSEIQRILDYSLCQNTWDKVERKEPLSPLDLSYLASKSPGKGLAYTVINGTLSFAHTRYVRMWIDGPVLKEMKGKRESPSGISSDIWTQWFKYGDMEIGPNGLLKTAGGYKFPWHLIGMGIVDNELHELSEANPLDHPQLPHAQSIADDSEEIFFGDTGVSKNPVELVTGWFTSWKESLAAGVVLILVVVLIYGVLRCFPVLCTTCRKPKWKKGVERSDSFEMRIFKPNNMRARV

SEQ ID NO: 19 - DNA, 1593 bp, Chandipura virus
atgacttcttcagtgacaattagtgtgatccttcttatctcctttattgccccatcatactcatctttgagtatagcatttccagaaaacaccaaattagattggaagccagtcacaaaaaacactagatactgccctatgggtggggaatggtttctagaaccagggttacaagaagaatctttcttgagctctacacccattggtgcgaccccctccaagtcagatggatttctctgtcatgcagccaagtgggtgacaacatgtgatttcagatggtatggacccaaatatattacgcattcaattcataatatcaaacctacccgatcagattgtgatacagcgcttgcatcatacaaatccgggacattagtgagccctggttttcccccagagtcttgtggttatgcttctgtgactgactccgagttcctggtgatcatgattacccctcatcacgtgggtgtggatgactacagaggacattgggtagatcctctttttgttggaggagaatgcgaccagtcttattgtgatactatccacaactcctcagtttggattcctgctgatcagactaagaagaacatttgcggccagtcctttaccccactgactgtgacggttgcttatgataaaaccaaagaaattgctgcaggcgcaatagtctttaagagcaaatatcactctcacatggaaggtgctcgaacttgcagattgagttattgcggtcggaacggaattaaattccccaatggagagtgggtcagcctggatgttaaaactaagatccaagagaaacctttacttcccttgtttaaagagtgtcctgctgggacagaggtgagatctactcttcaatccgatggggctcaagtcttgacctcggagattcagaggattttggattattccttgtgtcagaacacgtgggacaaggtagaacgcaaagagcctttgtctccattggatctcagctatttggcatctaaatccccggggaaaggtctggcatatacagtgataaatgggacattgtcatttgcccataccagatacgtgaggatgtggattgatggcccggtgttgaaagaaatgaaaggcaaaagggaatctcctagtgggatctcgagtgatatttggacccaatggttcaaatatggggatatggagataggcccaaacggcctcttaaagacagcaggagggtacaaattcccctggcatctgatcggtatgggaattgtggacaatgaactacacgagctcagtgaggcaaaccctttagaccatccacagctacctcatgctcagtctattgccgacgattcggaggagatcttctttggagacactggggtttccaagaatccagtagaactagttacagggtggttcactagctggaaagagagcttagctgccggtgttgttttgatattggtagttgtcctgatttatggtgtcctccgttgtttcccggtgttgtgtactacctgcagaaagcccaaatggaagaaaggggtagagaggtccgatagctttgagatgcggattttcaagcccaacaacatgagagccagagtatga

SEQ ID NO: 20 - protein, 820 aa, Curionopolis virus
MDLVRFSIALSVFLCYGTPPSQGQAIVSIKDSCEAKSAPWIPCEKFDYVKNATGSGIKCWIFCSRSGFYSKTGRFIRCIQGDPEAKYIKSCRRQIEKRGKEKMREGTRGKRKTSEPKEEGVRAKTDFTPDESRRLNNLTKVFRKVEDKDLNDFKKFILEKGLETKIKLANDGKISFRDPDCGENKDYPCHRIHQIIEGVNENIDYINEILSLKKMKEELRLRERESEEGEFPGLLNTTNRRGFLLHYPVELGNWSRLEDPSQIKCPSHHKDMLSNPRRLGKYNLDIIVRRPRIGTFETVVPGYICQGMQWTSTCNEMWYFVTYHDRAVHYITPNKLKCLQNIRAHKRGEHIKPYYPLEECNWNSETTKTVDYFMITPYSPEVDPFTLEFKSEIFPDRTSCRPGDEICVTDDDSKVWFPDEDDKLIARGHCPDETWDESHLTIHPEEMPENWEDPQSPWVSDYILKGVLFGEKRVKKSCLLEFCGTSGLLFEDGEWWELNVFSREKGRESLTKIFIEQEEIRRCNGTETRVGVAGKETDEKALLNAVLSKNAYERCKSARYRLIENKYLRLDDLSYINPRESVTWWAYRVRAGDDERTFKLEKTTGEYRYLQVPPSLEQHVTDCDGQENCSVSIGYYRGELINSSDWTRTGHDDVYVGVNGLLRKDTGNKTIVLYPPLMKEYQEIFSDSGESDDEAFIYKPDIHEKKGKPKEAEDEKDEKSKKNKTPIDDIKDWWSNIKGEWHLIKGILIGLFTFALLIGVVKLGVFIKSSFRKRRDDSIPEGKDEEIGIKMQSRRSRQNIYEEINEVSPTMTRRGRNIFN

SEQ ID NO: 21 - DNA, 2463 bp, Curionopolis virus
atggatcttgttcgattttcaattgcgttgtcagtcttcctatgctacggaacccccccatctcagggccaagcaatcgtttcgatcaaagatagttgcgaagctaagtcagctccctggatcccttgtgagaaatttgattatgtcaaaaatgctacggggtcaggaatcaaatgctggattttttgttctagatcaggtttttactcaaaaacagggaggtttattagatgcatccaaggagatccggaagcaaagtacataaaatcctgtcgaaggcagatagagaaaagagggaaagaaaagatgagagaaggaactaggggaaaaagaaaaacgtcggaaccaaaagaagaaggagtaagagctaaaacagattttactcctgatgagagcagaaggctgaacaacttgaccaaagtattcaggaaagtagaagataaggacctcaatgatttcaaaaagttcatattggaaaaaggattggaaacgaagattaagctagcaaatgatgggaagatctctttcagagaccctgactgcggagaaaacaaagactacccatgccacaggatccatcaaatcatagaaggggtcaatgaaaacatagactatataaatgagatcctaagcttaaaaaagatgaaggaagaattgaggttgagagaaagagaatcagaagaaggggagtttccaggccttctcaacacgactaatcgaagagggttccttcttcactaccccgtggaactaggaaattggtcaagactcgaagatcctagtcaaatcaaatgcccgtctcatcacaaggatatgcttagcaatcccagaagactggggaagtacaacttagacattatagtgaggaggcctagaatcggaacttttgaaacagtagtgcctggttatatatgccagggaatgcaatggacatctacttgcaatgagatgtggtattttgtcacttaccatgacagagcagtgcactacataacaccaaacaagctcaaatgtttacaaaacatcagagctcacaaaagaggagaacacataaaaccttattatcctctagaggaatgcaactggaattcagaaacaacaaaaacagtggattacttcatgatcacaccatactctcctgaagtagacccattcactctagaatttaaaagtgagatcttcccagacaggacgtcctgtcgtcccggagacgagatctgtgttaccgatgatgacagcaaagtctggttcccagacgaagatgacaagctgatcgcaaggggacactgtcctgatgaaacgtgggatgaatctcatctcactatacatccggaagagatgccggaaaattgggaagatcctcagtctccctgggtgagtgactacatactaaagggggtcttatttggagaaaagagagtcaaaaagagctgtctattagagttttgtggaacatctggactcttgtttgaagatggggaatggtgggagttaaatgttttcagcagagaaaaaggaagagaatcactgacgaagattttcatagagcaggaggagattcgacgatgcaacggaacagaaacccgtgtcggagtggctgggaaagaaactgatgaaaaagctttgttgaacgcagtgctgagcaaaaatgcctatgagaggtgcaagtctgctagatacagacttatagaaaacaagtacctcagattagatgacctcagctacataaatccaagagaatctgttacatggtgggcctacagagtgagagcaggagacgacgagagaacgttcaaattggaaaaaacgaccggagaatatcgttatctccaggttcccccttcattggagcaacatgtaacagactgtgatgggcaagaaaattgctctgtcagtatcggatactataggggagaactgataaactcatctgattggacgagaacaggacatgatgatgtctatgttggggttaatgggcttctacggaaagatacaggaaacaagacaatagttctataccctccactcatgaaagagtatcaggaaatattttcagatagtggggaatcagatgatgaggcatttatttataaaccagacatacatgagaagaaggggaagccaaaggaggcagaagatgaaaaagatgaaaagtcaaaaaagaacaagactcccattgatgacataaaagattggtggagcaacatcaagggggaatggcatctaatcaaaggaattctcatcggactgtttacattcgcgcttctgatcggagtcgtcaaactcggggttttcatcaaatcttcctttagaaagaggagagatgactccatacccgaggggaaagatgaagaaataggaatcaagatgcagtccaggaggtctagacagaatatttatgaagagatcaatgaagtgtcacccactatgacgagaagaggaagaaacatattcaattaa

SEQ ID NO: 22 - protein, 685 aa, Ekpoma-1 virus
MKKTTRRSSSETMILLIHLPVILTTLTKLISGDLINFPFHCTNLENIKYSNLSCPTVWETFKIKTGDKVERGSMCRPSLHTHDLEEGYLCYKDTWTTTCDESWYFSTEVKYKIIHEEVHDIDCLDALIEYKVGKLKAPFFPVATCYWASSTTESITFMMIKPHNAPLDPYSNRIVDPIIQADSGDNLKIYRTTFPKTRWIREVNTTLEERCNVATWECHDMTLYSGWLTHPSGAFKTSLRTGLVVDSQIMGHILLRDTCKMDFCGRRGFRFPDGGWWRLTTENEVSLQDFELNDTVVPKCDDRSRNHVGYTDLDYNPEKIALEQKSLLKTTMCREKLAELGQGKGMSLYDTTYLIPNAPGRYPAYYIYPVGLNKTLETQILKEKTISNPLTAKRKEHMPIMLYMAQCHYTLIEFPNLDSTGTLRYTSLEDPVGTILESGKNVSLADLGFEDINLDNTTCKGNDSDCFNTTTPKEPLLDRKFNMTNHTLPWRRYSKRELHHRVTYNGITHSPVGHWVQIPYGASLTANLPEHLIEKHSTHFFDHVTKQSIFERELQNGEISIDDLEQLIGRKTNHTDLPKKVRNWVQNAKESVVGIFREFGHTIRLGLSIVSFLIGLIISFKVWKKCRKNKKETQQQSRSSPIYRPQNIYELEEGPISPPPLARQREHDNSNIFRKTDPRNPFYSR

SEQ ID NO: 23 - DNA, 2058 bp, Ekpoma-1 virus
atgaaaaaaactacaaggcgttcgtcatctgaaaccatgattctactaattcatctccctgtaatcttaactactctcactaaattaatatccggagatcttatcaatttccctttccactgcactaatctagaaaacataaaatactctaatctgtcttgtcccacagtatgggaaacattcaagataaaaacaggagataaggtggaaagaggatcaatgtgccgtccttcgctacacacgcatgatctagaagaaggatatttgtgctataaggacacatggactacaacatgtgatgagtcatggtatttctcaacagaggtcaaatacaagatcattcatgaagaagtacatgacatagattgcttggatgccttaatagaatacaaggtcgggaagttgaaggcccctttctttcctgtcgctacatgttattgggcttctagcactactgagtcaatcaccttcatgatgattaaacctcataatgctcccttggacccttactcgaacagaatagttgacccaataatacaggcagatagcggagacaatttaaagatatataggacaacattccccaagacccgatggattagggaggtaaacacaacactcgaagaaagatgcaatgtcgcaacctgggaatgtcatgatatgacattatattcaggctggttgacacacccttcaggtgcatttaagacaagcctgaggacaggcctggtagtcgacagtcaaatcatgggacatattttactaagagatacttgcaaaatggatttttgcgggagaaggggatttagatttccggatggaggatggtggagattgactacagagaatgaagtgtcattgcaggattttgaactgaacgacaccgtcgtaccaaagtgtgacgacagaagtagaaaccatgtcggatataccgatttggactacaatccagaaaagattgcgttggagcaaaaatctctattgaaaacaacaatgtgtagagagaagctggcggaactaggccaaggcaaaggaatgagcctatatgacaccacatacctaatccccaacgctccaggtcggtacccagcttactatatataccctgttggtcttaacaagactctggaaacccagattctaaaggaaaagaccatctcgaatcctctgactgcaaagagaaaagaacacatgccgatcatgctttacatggctcaatgtcactataccctaattgagtttccaaaccttgacagtacaggaacattgagatacactagccttgaagaccccgtcggaacaatactggagtcagggaagaatgttagcctcgcagatctgggatttgaagatatcaaccttgacaacacaacgtgcaaaggaaatgactcagactgcttcaacacgaccactccaaaagaaccgctcctagataggaaattcaacatgacaaatcacaccctcccatggagaagatactccaaaagagaattacatcacagagtcacgtataatggaataactcatagtccagtgggtcattgggttcaaatcccatatggagcaagcctaacggcgaacctccctgaacatttaatagagaaacattccactcacttttttgatcatgtgactaaacaatctatatttgaaagagagttacagaatggagaaatatcaattgacgacttagaacaactaattgggaggaaaacaaatcatactgatttgcctaagaaagtaagaaactgggttcaaaatgcaaaggagagtgtcgtagggatttttcgagaatttggacatactatccggctaggactctctattgtatcattcttgattggattaatcatatcattcaaagtctggaaaaaatgcagaaagaacaagaaagagacacaacagcaatcaagatcttctcctatttatagacctcaaaacatctacgagttggaagaaggtcctataagtccgcctcctctcgccaggcaaagagaacacgacaacagcaatatcttcaggaagacagacccaagaaatcccttttattcgaggtaa

SEQ ID NO: 24 - protein, 630 aa, Ekpoma-2 virus
MQTMKKTHLLAFTIFGQILLASSLVVNLPLRCNGRKDLLVNSLKCPLPSTEVKVDGKVKVYEGDICRPQINAKDVEAGYLCHKDIYKAICDETWYFSATVKHEIEHAPISDIECIEGLTELKLGIVPNPQFPSVDCYWNARTEEKRTYIILTQHDPALDPYSNKIKDNVVDPDCDFNLCKTNFINTKWIRDKNTTEIERCDAKNWDCHPYKIYQGWISKSEMIGWGDPTQSYSYTGLVLDSHIYGHIPMSKLCHKTFCGKEGYLFPDKSWWQIRSKTPASPLFRELTLNGSRSAFPDCETIKTYGYAEVEEDESSEIIRESAEIRHEMCLETLSTLASGYEASFRDLMKFIPQRPGPGKAYSLNSNGKPSYYNYHWAGHPASSASIQEQDCYYYLVDIPKIQDDGILNITGIGNTDVCGKLLVNGSSMTLNSLGFKIDHHYDDHIVETGTDVHDEMNIKERMVWIKPDKIHPLLWVGPNGIVIDHQHKQIHFPVFSRGVDRIPHYWTQKHRVVKYRHATQLKIYKQYLDNPEKSNPYDFNAWTGRHVNRTEIPVAISNWFSGVKDTVFDKISKIGSWLKWSFYLCFIFVLFKGGLLVWNKYKTLRHQTKRTPKGKNSQDPEKLDIFGQTV

SEQ ID NO: 25 - DNA, 1893 bp, Ekpoma-2 virus
atgcagaccatgaaaaaaactcacttacttgcttttacaatttttggacagattctactggcttccagtctagtagttaaccttcctttgcgttgcaatggaagaaaggatttgttagtaaattcattaaaatgcccccttccaagcactgaagtaaaggttgatggaaaggtaaaagtgtatgaaggagacatatgcagaccccagataaacgctaaagatgtagaagcgggttatctctgccacaaagatatttataaggctatttgtgatgagacttggtatttctcagcaacagttaaacatgagatagaacatgctccaatatcagatatagagtgtatagaaggattaactgagttaaagcttggaatagtccctaacccacaatttccaagcgttgactgttactggaatgctagaactgaagagaagagaacgtacatcatcctaacccaacatgatcccgccctagacccatactcaaacaaaatcaaggacaatgtggtggatccagattgtgactttaatctgtgcaagaccaacttcatcaatacaaaatggattagagacaaaaacacgactgagatagagagatgcgacgcaaaaaactgggattgtcatccctacaaaatatatcaaggctggatcagcaaatcagagatgatcggctggggtgaccccacccagtcttactcatacacaggattggttttagattcacatatctacggacacattccaatgtccaaactatgccacaaaacattttgcggaaaagaaggttacctattccctgacaaatcctggtggcagatcagatcaaagactccagcaagtccattattcagggaattaaccttgaatggaagcagatctgcatttcctgactgtgagaccatcaaaacctacgggtatgctgaagtagaagaggatgaatcctcagaaataatccgagaaagtgcagaaatcaggcacgaaatgtgtctagagactctctcaacgttagcatctggatacgaagcatcctttagggatctaatgaaatttattccacagagacctggaccaggtaaagcatacagcctaaattcgaatggcaaaccgtcctattacaattaccactgggctggacacccagcatcaagtgccagcatccaggaacaagattgctattattacctggtggatatcccaaaaattcaagatgatggaattctgaatataacaggcataggaaacactgatgtttgtggtaaattgttggttaatgggtcatcaatgactttaaatagtctcggtttcaaaattgatcatcattatgatgatcatattgttgaaacagggacggatgtccatgatgaaatgaacatcaaagagaggatggtatggatcaagccagacaagattcatccgctcctatgggttggaccaaatgggatagtcattgatcaccagcacaagcaaatccactttccggtgttttctagaggtgttgacaggattcctcactattggactcagaagcacagagtggtaaaatacagacatgcaactcaactaaaaatatacaaacagtatctagacaaccccgagaaaagcaatccctacgatttcaatgcatggactggcagacatgtaaatcggaccgaaattcccgttgcaatctccaactggttctctggtgtcaaggatactgtgtttgacaaaataagcaagattggcagttggctgaaatggtcattttatttgtgttttatatttgtactattcaaaggaggtcttctagtctggaacaaatacaagacactacgtcatcaaacaaaaagaactccaaaaggaaaaaatagtcaagatcccgagaaactagatatttttgggcaaaccgtgtaa

SEQ ID NO: 26 - protein, 523 aa, Isfahan virus
MTSVLFMVGVLLGAFGSTHCSIQIVFPSETKLVWKPVLKGTRYCPQSAELNLEPDLKTMAFDSKVPIGITPSNSDGYLCHAAKWVTTCDFRWYGPKYITHSVHSLRPTVSDCKAAVEAYNAGTLMYPGFPPESCGYASITDSEFYVMLVTPHPVGVDDYRGHWVDPLFPTSECNSNFCETVHNATMWIPKDLKTHDVCSQDFQTIRVSVMYPQTKPTKGADLTLKSKFHAHMKGDRVCKMKFCNKNGLRLGNGEWIEVGDEVMLDNSKLLSLFPDCLVGSVVKSTLLSEGVQTALWETDRLLDYSLCQNTWEKIDRKEPLSAVDLSYLAPRSPGKGMAYIVANGSLMSAPARYIRVWIDSPILKEIKGKKESASGIDTVLWEQWLPFNGMELGPNGLIKTKSGYKFPLYLLGMGIVDQDLQELSSVNPVDHPHVPIAQAFVSEGEEVFFGDTGVSKNPIELISGWFSDWKETAAALGFAAISVILIIGLMRLLPLLCRRRKQKKVIYKDVELNSFDPRQAFHR

SEQ ID NO: 27 - DNA, 1572 bp, Isfahan virus
atgacttcagtcttattcatggttggtgtgctcttgggggcctttggttcaacccattgtagtattcaaatcgttttccccagtgaaacaaaactcgtatggaagccagtattaaaagggaccaggtactgtccacaaagtgcagaattaaatctggaacccgacttgaaaactatggcttttgacagcaaagttccaattggcataacgccttccaactcggatggctacctgtgtcatgctgccaaatgggtcacaacatgtgattttcgatggtatggaccgaagtacataactcactctgtccacagcttgagaccaacagtttctgattgtaaagcggccgtagaagcttacaatgctggtactctcatgtacccgggttttcctcctgaatcttgtggatatgcatctatcacggattctgaattttatgtcatgctagtaactccgcatcctgttggagtggatgattacagaggacactgggtggatccattgtttcctactagcgagtgcaattccaatttttgtgagactgttcacaatgccactatgtggatcccgaaagatcttaaaactcatgatgtttgttctcaggacttccagacgattagggtttccgtgatgtatcctcaaaccaaacccaccaagggggcagacttgacactgaaaagtaagttccatgctcacatgaaaggtgacagagtctgcaagatgaaattctgcaacaaaaatgggttgcgactgggaaacggagaatggattgaagttggggatgaggtcatgctcgataactcgaaactcttgagtttattcccagattgtttggttggttctgtggtaaaatccactttgctctcggaaggagttcaaacagcactgtgggagaccgacagactattagattactcattgtgccagaacacatgggaaaaaatcgatcgaaaagagccgctgtctgctgtggacctgagctatcttgcacctagatcacccggaaaggggatggcatacatcgttgccaatggatctttgatgtctgctcctgctagatacatcagagtttggattgacagtcccatacttaaggagataaaaggaaagaaagagtcagcctccggaattgacactgtcctttgggaacaatggctccccttcaatggaatggagttaggacctaatggattgatcaagacgaagtcaggttacaaatttccgctatatcttcttggaatgggcattgtagatcaagatcttcaagagttgtcctcagtgaaccctgtagaccacccacatgtaccaattgcccaggctttcgtttcagagggagaagaagtcttctttggggatacaggagtctctaaaaacccaatcgagctgatatctggctggttctcagattggaaagaaacagcagccgcattagggttcgctgcaatatctgtgatcttaattattggactaatgaggctgttgccactattatgcaggaggagaaagcaaaaaaaagttatctacaaagacgtagaattaaattcttttgatcctagacaagcttttcacagatga

SEQ ID NO: 28 - protein, 611 aa, Kamese virus
MSYLLVIILITINRLYAFSRDADHWYVRVPHDQSWFDNVITFPIDCKEPWQQITSQNLNCPSFNNISAEAKASFNLGTVFHPLASSRLTVDGYLCHKQSWISQCVETWYFSTTETNTISNLPITKSECEEAITMYEMGEYTNPFFPPFYCSWCSTQTDQKTFVIVEPHSVREDVYNGTFVDPLFVDGYCSADYCRTIHPDVLWVPRGQSMRKDVCNKGLWESGTVFGVLEERDEDLYYSIEEQLIRSSIYGVRRLEGACYRGVCNQFGIRFQSGEWWGLAGRDVVIWIKRILKQCARGQWISLSHDNHDERMAETQELMRTMLCENVKSRILSNDPVSPNDLNYLLPTNPGVGMAYRIFKRILLKGNHGGPTSELYMEQRHCMYRILHNVSRVINQTSGTWTIGQMFNGAPISINESVFERPSYLNNSARESGDGWFLLSYNGLIKYGNVLYTPSAVESSVEGLGFFHDRTSLLLLDSPKSVAVSSQMELVNNIYTSIFHSNTTSVFSKVEGAIRAAKNAVASYFSQLTNVAWWVGTGCIGIVALLIWRKCHCYDLLCKKTSRSADEISSKHIYDTIEMKPRTRVQNKASTPKLPPKRAHGKDLAHNYFQY

SEQ ID NO: 29 - DNA, 1836 bp, Kamese virus
atgagttacctattggttattattttgatcaccataaataggctttatgctttctccagagatgcagatcactggtatgttcgggtgccccatgaccagtcatggtttgataatgttataacgttcccgattgattgtaaagaaccttggcaacaaatcacctcccaaaatttaaattgcccctcattcaataacatcagtgcagaagcgaaagcttcgttcaacctggggactgtgtttcatcctcttgcaagcagtcgattaactgttgatggctatctctgtcataaacagtcatggatctcccaatgtgtggaaacatggtatttttcaacaacagaaacaaacaccatttcaaatcttccaataacaaaaagtgagtgtgaggaggccattacaatgtatgagatgggagaatacactaatccttttttccctccattctattgttcctggtgttccactcagaccgatcagaaaacatttgtaattgtggaaccgcactcagttagagaagatgtgtataatggtacatttgttgatcctttgtttgttgacggatattgttctgcagactattgccgcactatacaccctgatgtgttatgggtacctagaggtcaatctatgcgcaaagatgtttgcaataaaggtttatgggaatctggcactgtgtttggcgtcctggaggaaagagatgaggatttgtattatagtattgaggagcagctgatcagaagctcaatttatggggtaagaagattagaaggagcttgttacaggggggtgtgcaaccaattcggtataagatttcagtcaggagaatggtgggggttggctgggagagatgtggtcatctggatcaagagaattctaaaacaatgcgcaagaggtcaatggattagtttgagtcatgacaaccatgatgagcgcatggcggaaacacaagaattgatgcggactatgctgtgtgagaatgtaaagagtagaattctgagcaatgacccggtctccccgaatgatttaaattatctcctcccaactaatccaggtgttggtatggcatatcgaattttcaaacggatcttactgaaaggcaatcatggagggcctacctcagaactgtatatggagcaacggcattgcatgtacaggatactacacaatgtcagcagagtaataaaccaaacctcagggacctggactattgggcagatgttcaatggagcaccaattagtattaatgagagtgtatttgagagacccagttatttaaataactctgccagagaaagtggagacggatggttcttactctcctacaatgggcttattaagtatgggaacgtcctttacactcccagtgctgttgaatccagtgtggaaggcctaggttttttccatgacagaaccagtctactcttgctagattctcctaaatctgtcgcagtatcaagtcagatggagttggtgaataatatatacacctctattttccattcaaacacaacatctgttttctctaaggtggaaggtgctattagagctgccaagaatgctgttgcaagttacttctctcagctgaccaatgttgcttggtgggtaggaaccggttgtatagggattgtagccctattgatatggagaaaatgtcactgttatgatcttctgtgcaaaaaaacatccagatctgccgatgaaatatcttccaaacacatttatgataccatagaaatgaaaccccgaacccgtgttcaaaataaagcttcaactcctaaattaccacctaagagggctcatgggaaagacttagcccataattactttcaatactga

SEQ ID NO: 30 - protein, 611 aa, Kotonkan virus; identical to SEQ ID NO: 28

SEQ ID NO: 31 - DNA, 1920 bp, Kotonkan virus
atgaagagtctctattattcattgttcttgttattcaatgctaaaaatattataacctacagaattgcaaatttgcccttcaattgtgaaaacgaacattctatacctgttgaagccatagactgccctgtgaggagaaatgagcttaaagtagagaacctaaaacaaggtggagaacatagagtatgtaaacctaaactcagcacggatgatcatgttcaggggaaattatgccgtatacaacaatggaaaacaaagtgtacagaaacatggtactttacaacttacattgaatatgaggtggtagatgtaatgcccaacaaaatagaatgtgcaaaagagtgggagaggacaaaggctggatttcccataatccccttcttcccaccagctgtctgttattggaatgcagagaatgtaatatctgaaacttttgtaaccttagttgatcacccagtgttacaagatccttataacagcgaagtaattgaccccatattctatggcactcgatgttcaccgattaatagttttgattctcactggttttgcaagtcagttaataacctgataatgtggatgtcagacaaagatcaattgaggagtccgcattgtgatattaaaacatgggactgtattgttgtgaaggcatatgttgcatgggacgaagatcataatacacacaattatctaagaaacacaaaggtttgggaatcaccagatatcgggagagtgggtctctatgatgcatgtaaaaagaggttttgtggggttgatgggatcagattgaataatggagaatggtggtttttagaaagagaggaaaattattacggatttgactacagaggaatgaggaactgcagggccgaagaaactataggtgttagaacacatgtagatcgaacattgtttgaagaaattgacataaaattagaaatagaacatagtaaatgtatagatgttttaataaagttaagaagtgggattacaatatctccatttgaactaggttacttggcgccatcatcttatggaaagggctatgcatatcgctttgagcaggaaactaaaaacatatatcaatgtttccctaagattgagaaagtaccacagataaaatatataactgatgatttaaagaattgcaaagataataaaactagatatgcccgcactataacacaaaccaaaattggcaattataagcgagcattatgcaattacaaaaatgtattcataccagagactaaacaagatcaacaggcaggatatgacatcaaaatgtggacatttgctggtaccaatgacagcataaaagaacatatagagaacaatagctggtcaccatttaaatcgcaatcggggaataattataccatagggtggaacggaatgataaaactaaatacgggaagatatttgataaacacatatgcgttgcttgatggcttaatacatgaagctcaattatctgcattagaagtgaaatcttttcagcatcctgtataccagaattttgatgattttgcaaagtggttaaatggatcatcaatatacgaggaaagagaacttcttgatgacagtcatctagaaaggactgatgtaattaagtcagcaggagagaagatcaaaggaatttatcataatatagtgggttggttttcaggagtaacaagcatagtaaggtggatactctggggggtaggtgcaattgtaactgtatatgtgatcttgaaaattaggagagtgataaagaacaaacatgatgaaaaagataataagtcagaaataaaacaattctttgagaggttagggaaaataaagacacacaagggagataacaattctgtaccgaatattaaaggaaagagagacaagaaagaagatgagtatgagatgataaacttctatagttaa

SEQ ID NO: 32 - protein, 534 aa, Kwatta virus
MDKLIILTACLLGVVIASHDYYYFPVVQSKSFKKLPVGQLRCPPHSSEKPLSHKKIWGGYVLTQNIQTMPGTFVVKQRWGTTCTMNFWGVKTIRHHIIDEQILDARFTNITLKPVFPDEDCSWMTTATREITYYVGTKGELEYDISTGKTSDPVFGAFSCTEKLCYVDHRVVFIPDVAIAATSKGFKFVVFEISTDPDGVIRENSVIQSRDFPRMSLRKACVTEESVLGQRRLAFILRNGFFLVLEMGVKSGSHMLKKSTETLGSELILRASLRLSNDKFKGRDLSMLYTQEKISGAGSIDNLLNGFRVCDASDRSRIKQVGLGFNSLEQDERIMSRVDSLFCRVTLDRIRKCKKLTSVELGMFAQNYGGPGPVYRIKNDTLEVAQGIYKRIFWDPDTKNRLGYYVNETTEKEVNCPEWIKISEGFESCINGIIRYKNVTSHPLSPVNDLEQEEALFKEHFLEDVYHVPTQHLNPWAGWNPLHPPEIDRHFLGLKLPNIFGFMHNFEIYLVTFIVGLISLPLIIFCCRRKSSRY

SEQ ID NO: 33 - DNA, 1605 bp, Kwatta virus
atggacaagctcatcatcctcacagcatgtttgctaggagtcgtgattgcctcacatgattactattattttcctgtggtgcaatcaaagtcattcaagaaactgccggttggacaattgagatgtcctcctcattccagcgagaagcctttgtctcataagaaaatctgggggggttatgtattaacacagaacatacagacgatgcccggaacttttgtcgtgaaacaaaggtggggcacaacatgtacaatgaatttctggggagtcaaaacaattcgacatcatattatagatgagcaaatactagatgccagattcacaaacatcaccctaaagcctgtgttccctgatgaagattgctcttggatgacaaccgccacacgagaaatcacttactatgtggggacaaaaggagagctcgaatatgacatttcaactggaaaaacatcagacccagtcttcggcgctttttcatgcactgagaaactgtgctatgttgatcacagagtggtgtttatacctgatgtggccatagcagcaaccagcaaaggattcaaatttgttgtctttgagatctctaccgatccggatggtgtaataagggaaaactctgtaattcagtcacgtgacttccccagaatgtcattgaggaaagcatgtgtcacagaagagagtgtcttaggacagcgaagattggctttcatcttgaggaatgggttctttttagtgctggaaatgggagtaaagagtgggagtcacatgcttaaaaaatcaactgaaacattaggcagtgaattgattctgagggcctcccttcgattgagcaatgacaaattcaaaggtagagatctgtccatgttgtacacacaggagaagatttcaggggctggttccatagacaatttactcaatgggttcagggtctgtgatgctagtgacagatctaggataaaacaggtcggtcttggattcaattcgctagagcaagatgaacgcatcatgtctcgagtagactccttattttgtcgagtaactcttgacaggattcggaaatgtaagaagctcactagtgttgaattgggtatgttcgctcagaattatggtggtcccggtcctgtgtacagaataaagaatgacacattagaggtagctcaaggtatttacaagaggattttttgggatccagacacgaaaaaccgcttaggttattatgtgaatgagacaaccgagaaggaggttaactgtccggaatggatcaaaattagtgaaggatttgagagctgcatcaatggaatcattaggtacaagaatgtgacatcgcaccctctgtcaccagttaatgatctggaacaggaggaggcgttgttcaaagaacacttcttggaagatgtctatcatgtccctacacagcatctcaatccctgggcgggttggaaccccctgcatcctcctgagatagatcgtcatttcttaggactcaagctgccaaacatatttggatttatgcataattttgagatctacttagtgacattcatagttggattgattagtttgcctttgatcatcttttgttgtagaagaaagtcatctagatattaa

SEQ ID NO: 34 - protein, 573 aa, Le dantec virus
MWIITALICSFSINPTCLYPHGHEDSPTVRHGISRVLSGDAERNDDEHYHSPPLVLPLQNERTWKPANLSSLKCPEASHLGPDEHRVMEKWLVHRPKSSVLTKVEGSLCHKSRWLTRCEYTWYFSKTVSRKIEPMPPTKQECEEAIKRKEEGLLESLGFPPPACYWARTNDEENVQVDVTDHPMTYDPYSDGVVDNILVGGKCNQRECETVHDSTIWLETQKEKRPSQCEMDVEEQLELVSGIKRVGGSKSKAQRSVFVVGTNYPFMDATGACRLKYCSKSGMLLSNGLWFHITRKISPESNENSKFWLTLSDCSSDKQVGVLGEEYEIGKLQATMEDIMWDLDCFRTLEDLSHHKKVSMLDLFRLSRLTPGTGPAYKLVKGNLMVKEVQYVKAQRDQGELANPLCVAFMTESKNADRCIRYDEYDKEGPYKGQVMNGILINEGMVVFPHERFHLRQWDPEFIIKHEIKQVHHPVLGNYSSQIHDSLHESLIKDHSANLGDVMGNWVQVATSKFSWFFKEIEKFIIGGALLLIFILIALMVCRGGCCKVRRKAGGEKGGDSSGDEMNVSESIF

SEQ ID NO: 35 - DNA, 1730 bp, Le dantec virus
atgtggataatcaccgcactcatttgttccttcagcataaatccaacttgcctttatcctcatggtcatgaggattctcctactgtaagacatgggatttcccgtgttttgtctggagacgctgaacgaaatgatgatgagcattaccacagccctcccttggttttgcctttgcaaaatgaaagaacttggaaacccgctaatttgtcaagcttgaaatgccctgaagcttcccacttaggtcctgatgaacatagggtgatggagaaatggttagttcatagaccaaagtcatctgtcttaactaaagttgaaggttctttatgtcataaatcaagatggttgactagatgtgagtacacatggtatttttcgaaaactgtttccaggaagattgagccgatgcctcctactaaacaagagtgtgaagaagcgatcaaacggaaagaagagggattgttggagagtttaggtttccctccaccagcttgttactgggccagaacaaatgacgaagaaaatgtacaagtagatgtaactgaccaccccatgacatatgatccttacagtgatggagttgttgacaacatactagtaggtgggaaatgcaatcaaagagaatgtgagacagttcatgactctactatatggttggaaactcagaaagaaaagagaccatcacaatgtgaaatggacgtggaagaacagttagaattagtcagtgggattaaacgagtaggtggttcaaaatcaaaagcacagcgtagtgtcttcgttgttggcacaaattacccttttatggatgctacaggggcctgtagattaaaatattgcagtaagtcagggatgcttcttagcaatggattatggtttcatattacacgcaagatctcaccagagtcgaatgaaaacagtaagttttggttgacgctatctgattgttcatctgataaacaagttggggttttaggagaggaatatgagattgggaaactccaagcaacaatggaggatatcatgtgggacttagattgttttaggacgttagaggatttatcccatcacaaaaaggtcagcatgttagatttgtttagactttctagattaacaccaggcacaggtccagcttacaagttagttaagggaaatcttatggttaaggaagtacagtatgtgaaagctcagagagatcaaggagaattagcaaatcctctatgtgttgcttttatgacggagtcaaaaaatgcagacagatgtattcgttatgatgagtatgacaaagaaggtccctataaaggccaggtaatgaatggaatattgattaatgaggggatggttgtcttccctcatgagagatttcacctgaggcaatgggatccagaattcattatcaagcatgagataaaacaagttcatcaccctgtattaggaaattattcaagtcagattcatgattctctacatgaaagccttattaaagatcacagtgcaaatttgggagatgtaatgggcaactgggttcaagtagctacatctaaattttcttggttcttcaaagaaatagaaaagttcatcattggaggagcactgttgttgatatttattttaattgcactaatggtgtgtagaggtggatgctgtaaagtaagaagaaaggcaggtggggaaaagggaggagactcttcaggagatgaaatgaatgtaagcgaaagcatcttttaaaaccatga

SEQ ID NO: 36 - protein, 524 aa, Rabies virus
MVPQVLLFVLLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTIESLIIISPSVTDLDPYDKSLHSRVFPGGKCSGITVSSTYCSTNHDYTIWMPENPRPRTPCDIFTNSRGKRESNGNKTCGFVDERGLYKSLKGACRLKLCGVLGLRLMDGTWVATQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGLILGPDDHVLIPEMQSSLLQQHMELLESSVIPLMHPLADPSTVFKEGDEAEDFVEVHLPDVYKQISGVDLGLPNWGKYVLMTAGAMIGLVLIFSLMTWCRRANRPESKQRSFGGTGGNVSVTSQSGKVIPSWESYKSGGETRL

SEQ ID NO: 37 - DNA, 1575 bp, Rabies virus
atggttcctcaggttcttttgtttgtactccttctgggtttttcgttgtgtttcgggaagttccccatttacacgataccagacgaacttggtccctggagccctattgacatacaccatctcagctgtccaaataacctggttgtggaggatgaaggatgtaccaacctgtccgagttctcctacatggaactcaaagtgggatacatctcagccatcaaagtgaacgggttcacttgcacaggtgttgtgacagaggcagagacctacaccaactttgttggctatgtcacaaccacattcaagagaaagcatttccgccccaccccagacgcatgtagagccgcgtataactggaagatggccggtgaccccagatatgaagagtccctacacaatccataccccgactaccactggcttcgaactgtaagaaccaccatagagtccctcattatcatatccccaagtgtgacagatttggacccatatgacaaatcccttcactcgagggtcttccctggcggaaagtgctcaggaataacggtgtcctctacctactgctcaactaaccatgattacaccatttggatgcccgagaatccgagaccaaggacaccttgtgacatttttaccaatagcagagggaagagagaatccaacgggaacaagacttgcggctttgtggatgaaagaggcctgtataagtctctaaaaggagcatgcaggctcaagttatgtggagttcttggacttagacttatggatggaacatgggtcgcgacgcaaacatcagatgagaccaaatggtgccctccagatcagttggtgaatttgcacgactttcgctcagacgagattgagcatctcgttgtggaggagttagtcaagaaaagagaggaatgtctggatgcattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtaaacggggtgtttttcaatggtttaatattagggcctgacgaccatgtcctaatcccagagatgcaatcatccctcctccagcaacatatggagttgctggaatcttcagttatccccctgatgcaccccctggcagacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagaagagccaatcgaccagaatcgaaacaacgcagttttggagggacaggggggaatgtgtcagtcacttcccaaagcggaaaagtcataccttcatgggaatcatataagagtggaggtgagaccagactgtga

SEQ ID NO: 38 - DNA, 5670 bp, Artificial Sequence
  Synthetic plasmid
  misc_feature: plasmid with gene for human L-Selectin
  misc_feature (985)..(1004): n is a, c, g, t or u
  misc_feature (2163)..(2174): n is a, c, g, t or u
  misc_feature (2202)..(2202): n is a, c, g, t or u
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgcgaattcnnnnnnnnnnnnnnnnnnnnatgggctgcagaagaactagagaaggaccaagcaaagccatgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaannnnnnnnnnnnagatctggtaccgatatcaagcttgtcngactctagattgcggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatatt

SEQ ID NO: 39 - protein, 385 aa, Homo sapiens
MGCRRTREGPSKAMIFPWKCQSTQRDLWNIFKLWGWTMLCCDFLAHHGTDCWTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYNPLFIPVAVMVTAFSGLAFIIWLARRLKKGKKSKRSMNDPY

SEQ ID NO: 40 - DNA, 1158 bp, Homo sapiens
atgggctgcagaagaactagagaaggaccaagcaaagccatgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaa

SEQ ID NO: 41 - protein, 496 aa, Machupo arenavirus
MGQLISFFQEIPVFLQEALNIALVAVSLIAVIKGIINLYKSGLFQFIFFLLLAGRSCSDGTFKIGLHTEFQSVTLTMQRLLANHSNELPSLCMLNNSFYYMRGGVNTFLIRVSDISVLMKEYDVSIYEPEDLGNCLNKSDSSWAIHWFSNALGHDWLMDPPMLCRNKTKKEGSNIQFNISKADDARVYGKKIRNGMRHLFRGFHDPCEEGKVCYLTINQCGDPSSFDYCGVNHLSKCQFDHVNTLHFLVRSKTHLNFERSLKAFFSWSLTDSSGKDMPGGYCLEEWMLIAAKMKCFGNTAVAKCNQNHDSEFCDMLRLFDYNKNAIKTLNDESKKEINLLSQTVNALISDNLLMKNKIKELMSIPYCNYTKFWYVNHTLTGQHTLPRCWLIRNGSYLNTSEFRNDWILESDHLISEMLSKEYAERQGKTPITLVDICFWSTIFFTASLFLHLVGIPTHRHLKGEACPLPHKLDSFGGCRCGKYPRLKKPTIWHKRH

SEQ ID NO: 42 - DNA, 1491 bp, Machupo arenavirus
atggggcagcttatcagcttctttcaggagattcctgtttttctacaggaagctctgaacatcgctttagtggctgttagtctcatagctgtcatcaaaggcatcattaacctttacaaaagtggtctcttccagttcatcttctttctcctcctagcagggaggtcctgctcggatggcacattcaaaataggcctacacactgagttccagtcagtcacccttaccatgcagagacttttagctaaccattcaaatgagctcccatctctctgcatgcttaacaatagtttttattatatgaggggaggtgtgaacaccttcctgattcgtgtttctgatatttcagtcctcatgaaggagtatgatgtatcaatctatgaaccagaagaccttggaaattgtcttaacaagtctgactcaagctgggctattcattggttctcaaatgctttgggacatgactggcttatggatcctccaatgctatgtagaaacaagacaaagaaggagggatctaacattcaattcaacatcagcaaagctgatgatgccagagtgtatggaaagaagataagaaatggtatgaggcatctcttcaggggcttccatgacccgtgtgaggaagggaaagtgtgctacctgaccatcaatcagtgtggtgaccccagttcctttgactactgtggcgtgaatcatctttccaaatgtcagtttgaccatgtgaacacccttcatttccttgtgagaagtaagacacatctcaactttgagaggtctttgaaagcatttttctcatggtctctgacagactcctcaggaaaggacatgccaggaggttattgtctagaggaatggatgttgatagcagccaaaatgaaatgtttcggaaacactgctgttgctaaatgtaatcaaaatcatgactcagagttctgtgatatgctgaggctattcgactataacaagaatgcaataaagaccctcaatgatgaatcaaagaaagaaatcaatcttctaagccagacagtgaatgccttaatctcagataatttgttaatgaagaataaaattaaagagctaatgagcatcccttattgtaattacacaaagttttggtatgtcaatcataccctgacagggcagcacactcttccaagatgttggttgataaggaatggaagttatcttaacacttctgaattcaggaatgactggattttagagagtgatcacctcatctcagagatgttaagtaaggaatatgctgaaaggcaaggcaaaaccccaatcacattagttgatatttgtttctggagcacaattttcttcacagcatcattgttccttcatctagtcggaatacccacccatcgacacctcaaaggcgaagcctgtcctttgcctcataagctggacagcttcggaggttgtagatgtggcaaatatcccagattgaagaaacccaccatctggcacaaaagacattaa

SEQ ID NO: 43 - protein, 511 aa, Cocal virus
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVAVRYRYQGSNNKRIYNDIEMSRFRK

SEQ ID NO: 44 - DNA, 6507 bp, Artificial Sequence; identical to SEQ ID NO: 1
  Synethic Plasmid
  misc_feature: plasmid with a sequence from Indiana vesiculovirus

SEQ ID NO: 45 - DNA, 6805 bp, Artificial Sequence
  Synthetic plasmid
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgcgaattcggcacgaggcagcacagcacactccctttgggcaaggacctgagacccttgtgctaagtcaagaggctcaatgggctgcagaagaactagagaaggaccaagcaaagccatgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaaatcgcccttggtgaaagaaaattcttggaatactaaaaatcatgagatcctttaaatccttccatgaaacgttttgtgtggtggcacctcctacgtcaaacatgaagtgtgtttccttcagtgcatctgggaagatttctacctgaccaacagttccttcagcttccatttcgcccctcatttatccctcaacccccagcccacaggtgtttatacagctcagctttttgtcttttctgaggagaaacaaataagaccataaagggaaaggattcatgtggaatataaagatggctgactttgctctttcttgactcttgttttcagtttcaattcagtgctgtacttgatgacagacacttctaaatgaagtgcaaatttgatacatatgtgaatatggactcagttttcttgcagatcaaatttcacgtcgtcttctgtatactgtggaggtacactcttatagaaagttcaaaaagtctacgctctcctttctttctaactccagtgaagtaatggggtcctgctcaagttgaaagagtcctatttgcactgtagcctcgccgtctgtgaattggaccatcctatttaactggcttcagcctccccaccttcttcagccacctctctttttcagttggctgacttccacacctagcatctcatgagtgccaagcaaaaggagagaagagagaaatagcctgcgctgttttttagtttgggggttttgctgtttccttttatgagacccattcctatttcttatagtcaatgtttcttttatcacgatattattagtaagaaaacatcactgaaatgctagctgcaagtgacatctctttgatgtcatatggaagagttaaaacaggtggagaaattccttgattcacaatgaaatgctctcctttcccctgcccccagaccttttatccacttacctagattctacatattctttaaatttcatctcaggcctccctcaaccccaccacttcttttataactagtcctttactaatccaacccatgatgagctcctcttcctggcttcttactgaaaggttaccctgtaacatgcaattttgcatttgaataaagcctgctttttaagtgttaaaaaaaaaaaaaaaaaactcgactctagattgcggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 46 - DNA, 7411 bp, Artificial Sequence
  Synethetic plasmid
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggtcgaggatatcggatctagatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctaggatcaaggtcaggaacagagaaacaggagaatatgggccaaacaggatatctgtggtaagcagttcctgccccgctcagggccaagaacagttggaacaggagaatatgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatcgatgaattcgagctcggtacccggggatcccgggtgatcagtcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaactcgacgggcccgcggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 47 - DNA, 10195 bp, Artificial Sequence
  Synthetic plasmid
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcggtacgtaccatgaggacagctaaaacaataagtaatgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttagtgatacttgtgggccagggcattagccacaccagccaccactttctgataggcagcctgcactggtggggtgaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaataccctgttacttctccccttcctatgacatgaacttaaccatagaaaagaaggggaaagaaaacatcaagggtcccatagactcaccctgaagttctcaggatccacgtgcagcttgtcacagtgcagctcactcagctgggcaaaggtgcccttgaggttgtccaggtgagccaggccatcactaaaggcaccgagcactttcttgccatgagccttcaccttagggttgcccataacagcatcaggagtggacagatccccaaaggactcaaagaacctctgggtccaagggtagaccaccagcagcctaagggtgggaaaatagaccaataggcagagagagtcagtgcctatcagaaacccaagagtcttctctgtctccacatgcccagtttctattggtctccttaaacctgtcttgtaaccttgataccaacctgcccagggcctcaccaccaacggcatccacgttcaccttgtcccacagggcagtaacggcagacttctcctcaggagtcaggtgcaccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacacggtatcgataagcttgatatcgaattcctgcagcccccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatacgtatatgtgtatatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaaggaaaaactgaagcttatttaatcagagatgaggatgctggaagggatagagggagctgagcttgtaaaaagtatagtaatcattcagcaaatggttttgaagcacctgctggatgctaaacactattttcagtgcttgaatcataaataagaataaaacatgtatcttattccccacaagagtccaagtaaaaaataacagttaattataatgtgctctgtcccccaggctggagtgcagtggcacgatctcagctcactgcaacctccgcctcccgggttcaagcaattctcctgcctcagccaccctaatagctgggattacaggtgcacaccaccatgccaggctaatttttgtactttttgtagaggcagggtatcaccatgttgtccaagatggtcttgaactcctgagctccaagcagtccacccacctcagcctcccaaagtgctgggattacaggtgtgagacaccatgcccagattttccatatttaatagaggtatttatgggatgggggaaaagaatgtttctctcactgtggattattttagagagtggagaatggtcaagatttttttaaaaattaagaaaacataagttggaccttgagaaatgaaaatttatttttttgttggaggatacccattctctatctcccatcagggcaagctgtaaggaactggctaagacacagtgagacagagtgacttagtcttagaggccccactggtacgacggtcaccaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcagtcttgtcatggcaaaataaagataataatagtgtttttttatggagttagcgtgaggatggaaaacaatagcaaaattgattagactataaaaggtctcaacaaatagtagtagattttatcatccattaatccttccctctcctctcttactcatcccatcacgtatgcctcttaattttcccttacctataataagagttattcctcttattatattcttcttatagtgattctggatattaaagtgggaatgaggggcaggccactaacgaagaagatgtttctcaaagaagcgggggatccactagttctagagcggccaaatggcggccgtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttgg

SEQ ID NO: 48 - DNA, 4174 bp, Artificial Sequence
  Synthetic plasmid
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgatgtacgggccagatatacgcgtatctgaggggactaggtgtgtttaggcgaaaagcggggcttcggttgtacgcggttaggagtcccctcaggattagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagtcgattaggcatctcctatggcaggaagaagcggagacagcgacgaagacctcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagctatagcagtagctgaggggacagatagggttatagaagtagtacaagaagcttggcactggccgtcgttttacatgatctgagcctgggagatctctggctaactagggaacccactgcttaagcctcaataaagcttgccttgagtgcttcaagtagtgtgtgcccgtctgttgtgtgactctggtaactagagatcaggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 49 - DNA, 8895 bp, Artificial Sequence
  Synthetic plasmid
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattccggagcggccgcaggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttccgcggaattcaccccaccagtgcaggctgcctatcagaaagtggtggctggtgtggctaatgccctggcccacaagtttcactaagctcgcttccttgctgtccaatttctattaaaggttccttggttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagtttggctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 50 - DNA, 21 bp, Artificial Sequence
  Synthetic Polynucleotide
  misc_feature: Forward Primer
acttgaaagcgaaagggaaac

SEQ ID NO: 51 - DNA, 21 bp, Artificial Sequence
  Synthetic Polynucleotide
  misc_feature: Reverse Primer
cgcacccatctctctccttct

SEQ ID NO: 52 - DNA, 24 bp, Artificial Sequence
  Synthetic Polynucleotide
  misc_feature: Probe
  misc_feature (1)..(1): 6FAM
  misc_feature (24)..(24): TAMRA
agctctctcgacgcaggactcggc

SEQ ID NO: 53 - DNA, 24 bp, Artificial Sequence
  Synthetic Polynucleotide
  misc_feature: Forward Primer
ctctgagctattccagaagtagtg

SEQ ID NO: 54 - DNA, 18 bp, Artificial Sequence
  Synthetic Polynucleotide
  misc_feature: Reverse Primer
cagtgagcgcgcgtaata

SEQ ID NO: 55 - DNA, 25 bp, Artificial Seuqnce
  Synthetic Polynucleotide
  misc_feature: Probe
  misc_feature (1)..(1): 6FAM
  misc_feature (25)..(25): TAMRA
gacgtacccaattcgccctatagtg

SEQ ID NO: 56 - DNA, 1539 bp, Cocal virus
atgaatttcctactcttgacatttattgtgttgccgttgtgcagccacgccaagttctccattgtattccctcaaagccaaaaaggcaattggaagaatgtaccatcatcttaccattactgcccttcaagttcggatcaaaactggcacaatgatttgcttggaatcacaatgaaagtcaaaatgcccaaaacacacaaagctattcaagcagacgggtggatgtgtcatgctgccaaatggatcactacctgtgactttcgctggtacggacccaaatacatcactcactccattcattccatccagcctacttcagagcagtgtaaagaaagcatcaagcaaacaaaacaaggtacttggatgagtcctggcttccctccacagaactgcgggtatgcaacagtaacagactctgtcgctgttgtcgtccaagccactcctcatcatgtcttggttgatgaatatactggagaatggatcgactctcaattccccaacgggaaatgtgaaaccgaagagtgcgagaccgtccacaactctaccgtatggtactctgactacaaagtaactggattatgtgacgcaactctggtagacacagagatcaccttcttctctgaagatggcaaaaaagaatctatcgggaagcccaacacaggctataggagcaactacttcgcttatgagaaaggggacaaagtatgtaaaatgaactactgcaagcatgcgggtgtgaggttgccttccggggtttggtttgagtttgtggatcaggatgtctacgccgccgccaaacttccagaatgccccgttggtgccactatctccgctccgacacagacctctgttgacgtaagtctcattctagatgtagagagaattttagattactctctgtgtcaagagacatggagcaagatccggtccaaacagccagtatcccctgttgaccttagttacttggcccccaagaatcctgggaccggaccggcattcacaatcatcaatggcactctgaagtactttgagaccagatacattcggattgatatagacaatccaatcatctccaagatggtggggaaaataagtggcagtcaaacagaacgagaattgtggacagagtggttcccctacgagggtgtcgagatagggccaaatgggattctcaaaacccctacaggatacaaattcccactcttcatgataggacacgggatgctagattccgacttgcacaagacgtcccaagcagaggtctttgaacatcctcaccttgcagaagcaccaaagcagttgccggaggaggagactttattttttggtgacacaggaatctccaaaaatccggtcgaactgattgaagggtggtttagtagttggaagagcactgtagtcacctttttctttgccataggagtatttatactactgtatgtagtggccagaattgtgatcgcagtgagatacagatatcaaggctcaaataacaaaagaatttacaatgatattgagatgagcagatttagaaaatga

SEQ ID NO: 57 - protein, 529 aa, Piry virus
MDLFPILVVVLMTDTVLGKFQIVFPDQNELEWRPVVGDSRHCPQSSEMQFDGSRSQTILTGKAPVGITPSKSDGFICHAAKWVTTCDFRWYGPKYITHSIHHLRPTTSDCETALQRYKDGSLINLGFPPESCGYATVTDSEAMLVQVTPHHVGVDDYRGHWIDPLFPGGECSTNFCDTVHNSSVWIPKSQKTDICAQSFKNIKMTASYPSEGALVSDRFAFHSAYHPNMPGSTVCIMDFCEQKGLRFTNGEWMGLNVEQSIREKKISAIFPNCVAGTEIRATLESEGARTLTWETQRMLDYSLCQNTWDKVSRKEPLSPLDLSYLSPRAPGKGMAYTVINGTLHSAHAKYIRTWIDYGEMKEIKGGRGEYSKAPELLWSQWFDFGPFKIGPNGLLHTGKTFKFPLYLIGAGIIDEDLHELDEAAPIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

(19) United States
(12) Patent Application Publication
(10) Pub. No.: US 2021/0139932 A1
(43) Pub. Date: May 13, 2021

(54) IMPROVED LENTIVIRUSES FOR TRANSDUCTION OF HEMATOPOIETIC STEM CELLS

(71) Applicant: BIOMARIN PHARMACEUTICAL INC., Novato, CA (US)

(72) Inventors: Michael Lochrie, Hayward, CA (US); Wes Yonemoto, Novato, CA (US); Ramya Ankata, Novato, CA (US); Jac Michael, Novato, CA (US)

(21) Appl. No.: 16/610,819

(22) PCT Filed: May 3, 2018
(86) PCT No.: PCT/US2018/030956

§ 371 Date: Nov. 4, 2019

Related U.S. Application Data
(60) Provisional application No. 62/500,874, filed on May 3, 2017.

(51) Int. Cl. C12N 15/86; C12N 7/00; C07K 14/805; C12N 5/0789; A61K 35/28

(52) U.S. Cl. CPC C12N 15/86; C12N 7/00; C07K 14/805; C12N 5/0647; C12N 2510/00; C12N 2740/15021; C12N 2740/15043; C12N 2810/6081; A61K 35/28

(57) ABSTRACT

Recombinant viruses, comprising a lentiviral vector carrying a heterologous transgene, packaged in an envelope containing at least one heterologous envelope protein, are described. Also described are methods of producing these recombinant viruses and methods of using these viruses to deliver genes to selected target cells. These recombinant viruses are particularly useful for transducing a hematopoietic stem cells, in particular CD34+ cells.

### CROSS-REFERENCE TO RELATED APPLICATIONS

This application claims benefit of and priority to U.S. Ser. No. 62/500,874, filed on May 3, 2017, which is incorporated herein by reference in its entirety for all purposes.

### FIELD OF INVENTION

The field of this invention is in the area of improving lentiviral transduction of hematopoietic stem cells, preferably human CD34+ cells.

### BACKGROUND

Recombinant lentiviruses are useful for delivering heterologous transgenes (i.e., genes that are not native to the lentivirus) to hematopoietic stem cells in order to treat genetic diseases such as adenosine deaminase deficiency (Farinelli, et al, 2014), β-thalassemia, sickle cell disease (Negre et al., 2016), severe combined immune deficiencies, metachromatic leukodystrophy, adrenoleukodystrophy, Wiskott-Aldrich syndrome, chronic granulomatous disease (Booth et al., 2016), and several lysosomal storage disorders (Rastall, et al., 2015).

However, the efficiency at which lentiviruses can transduce primary hematopoietic stem cells (e.g., huamnCD34+ cells) is not as good as for transformed cell lines such as 293T cells. There have been many hypotheses proposed to explain this efficiency difference. Nearly all recombinant lentiviruses made to date contain the envelope protein from Indiana strain of vesicular stomatitis virus (VSV). One observation is that resting CD34 cells express very low levels of the main receptor for VSV (Indiana) (Amirache, et al., 2014), which is the low density lipoprotein receptor (Finkelshtein, et al, 2013). Cytokine stimulation of CD34 cells, which is needed to maintain the viability and stimulate cell division of CD34+ cells that have been frozen, upregulates the low density lipoprotein receptor and results in a modest increase in transduction by lentiviruses containing the envelope protein from the Indiana strain of VSV. Therefore, recombinant lentiviruses that comprise envelope proteins that do not use the low density lipoprotein receptor as a receptor to enter cells, and methods to enhance transduction by VSV envelope proteins, would be useful.

New enveloped viruses are constantly being discovered. In particular in recent years viral sequences have been identified by massively parallel (or “deep”) nucleic acid sequencing methods. Many of those sequences are from viruses with unknown biologies. Therefore they provide an opportunity to discover envelope proteins with useful properties such as improved transduction of hematopoietic stem cells.

Another approach for improving transduction of hematopoietic stem cells would be to identify non-viral proteins (i.e., cellular proteins) that can be assembled with lentiviruses and allow, for example, enhanced binding to CD34+ cells or other subsets of cells thought to be long term repopulating hematopoietic stem cells such as CD133+ cells. Single-chain antibodies that bind CD133 and are fused to the measles virus envelope protein have been used for this purpose (Brendel, et al, 2015). Such lentiviruses with engineered and fused envelope proteins can have better selectivity for target cells but that is often at the expense of reduced virus production. Other proteins that bind proteins on the surface of CD34 cells might be useful, especially if they are transmembrane proteins which may allow them to be more easily incorporated into the membrane of a lentivirus. For example CD52 is expressed in CD34+ cells (Klabusay, M., et al, 2007) and SIGLEC10 is a known ligand for CD52 (Bandala-Sanchez E., et al., 2013). CD34 is expressed on CD34+ cells and L-selectin is a known ligand that binds CD34 (Nielsen, J. S., et al., 2009). Preferably such proteins would not be expressed in virus producer cells (typically human 293T cells) because envelope-receptor interactions within virus producer cells are thought to be a cause of toxicity in virus producer cells which necessitates the use of transient transfection systems for producing virus and has hindered development of scalable stable lentivirus producing cell lines.

The difficulties associated with the efficiency at which lentiviruses can transduce primary human hematopoietic stem cells has necessitated improvements in the area of lentiviral transduction of human hematopoietic stem cells required for gene therapy applications.

### SUMMARY

Described in the present application are alternate vesiculovirus envelope proteins and/or arenavirus envelope proteins that enable more efficient transduction of hematopoietic stem cells, such as human CD34+ cells by recombinant lentiviruses than the prototypical VSV-G (Indiana strain) pseudotyped lentivirus, as well as methods for improving transduction of human CD34+ cells by recombinant lentiviruses by expression of a ligand for binding to human CD34+ cells, such as L-selectin, in lentivirus-producing cells. Accordingly, in various aspects, the invention(s) contemplated herein may include, but need not be limited to, any one or more of the following embodiments:

In one aspect, the invention provides a recombinant lentivirus capable of transducing a hematopoietic stem cell, said recombinant lentivirus comprising i) a heterologous transgene, ii) a viral envelope protein, and iii) a protein that is a ligand for binding to CD34+ cells. In one embodiment, the recombinant lentivirus comprise a vesiculovirus envelope protein. For example, the vesiculovirus envelope protein originates from a species of vesiculovirus selected from the group consisting of Vesicular Stomatitis Virus G (VSV-G), Morreton, Maraba, Cocal, Alagoas and Carajas. The VSV-G envelope protein may originate from the Arizona, Indiana or New Jersey strains of VSV-G.

In addition, the recombinant lentivirus comprises a viral envelope protein comprising at least 70%, 75%, 80%, 85%, 90%, 95%, 96%, 97%, 98% or 99% sequence identity to the amino acid sequence of a viral envelope protein disclosed herein as SEQ ID NO:2, 4, 6, 8, 10, 12, 14, 16, 18, 20, 22, 24, 26, 28, 30, 32, 34, 36 or 43 when the sequence comparison is carried out over the entire length of the two sequences. In one or more additional embodiments, the amino acid sequence of said viral envelope protein comprises, consists essentially of, or consists of the amino acid sequence disclosed herein as SEQ ID NO:2, 4, 6, 8, 10, 12, 14, 16, 18, 20, 22, 24, 26, 28, 30, 32, 34, 36, or 43.

In another embodiment, the recombinant lentivirus comprises a viral envelope protein comprising at least one of the 31 amino acids within the CD34 cell transduction determinant shown in FIG. 4 at its respective location. In addition, the viral envelope protein comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or all 31 of the 31 amino acids within the CD34 cell transduction determinant shown in FIG. 4 at their respective locations.

In another embodiment, the recombinant lentivirus may comprise an arenavirus envelope protein. For example the arenavirus envelope protein may originate from a Machupo, Junin, Ocozocoautla, Tacaribe, Guanarito, Amapar, Cupixi, Sabia or Chapre virus. In addition, the recombinant lentivirus comprises a viral envelope protein comprising an amino acid sequence having at least 70%, 75%, 80%, 85%, 90%, 95%, 96%, 97%, 98% or 99% sequence identity to the amino acid sequence of SEQ ID NO:41, when the sequence comparison is carried out over the entire length of the two sequences. In one or more additional embodiments, the amino acid sequence of said viral envelope protein comprises, consists essentially of, or consists of the amino acid sequence disclosed herein as SEQ ID NO:41.

Any of the recombinant lentivirus of the disclosure are capable of transducing a hematopoietic stem cell, such as a human CD34+ cell.

Any of the recombinant lentivirus of the disclosure, further comprise a vector; and wherein the vector comprises said heterologous transgene operably linked to a promoter.

Any of the recombinant lentivirus of the disclosure comprise a self-activating (SIN) LTR.

In another embodiment, the heterologous transgene of the recombinant lentivirus encodes a human protein. Optionally, the heterologous transgene encodes a human hemoglobin protein. In a further embodiment, the recombinant lentivirus also comprises a protein that is a ligand for binding to CD34+ cells. Optionally, the protein that is as a ligand for binding to CD34+ cells is present on the surface of said recombinant lentivirus. The protein that is as a ligand for binding to CD34+ cells comprises an amino acid sequence having at least 70%, 75%, 80%, 85%, 90%, 95%, 96%, 97%, 98% or 99% sequence identity to the amino acid sequence of SEQ ID NO:39, when the sequence comparison is carried out over the entire length of the two sequences. Optionally, the protein that is a ligand for binding to human CD34+ cells comprises, consists essential of or consists of the amino acid sequence of SEQ ID NO:39.

In a further embodiment, any of the recombinant lentivirus of the disclosure is produced by a cell having a concentration ratio of vector expressing the envelope protein and the vector expressing L-selectin ranging from 1:2 to 1:5. In addition, any of the recombinant lentivirus of the disclosure wherein the concentration ratio of the envelope protein and L-selectin ranges from 1:2 to 1:5.

In another aspect of the present invention, a method is provided for introducing a heterologous transgene into a hematopoietic stem cell comprising the step of transducing said stem cell with a recombinant lentivirus that comprises (i) said heterologous transgene and (ii) a viral envelope protein and (iii) a protein that is a ligand for binding to CD34+ cells. Any of the recombinant lentivirus of the disclosure may be used in the methods of introducing a heterologous transgene to a hematopoietic stem cell. In any of the methods, the hematopoietic stem cell is a human hematopoietic stem cell, such as a human CD34+ cell.

In one embodiment, the method comprise a recombinant lentivirus comprising a vesiculovirus envelope protein. For example, the vesiculovirus envelope protein originates from a species of vesiculovirus selected from the group consisting of Vesicular Stomatitis Virus G (VSV-G), Morreton, Maraba, Cocal, Alagoas and Carajas.

In addition, the methods comprise a recombinant lentivirus comprising a viral envelope protein comprising at least 70%, 75%, 80%, 85%, 90%, 95%, 96%, 97%, 98% or 99% sequence identity to the amino acid sequence of a viral envelope protein disclosed herein as SEQ ID NO:2, 4, 6, 8, 10, 12, 14, 16, 18, 20, 22, 24, 26, 28, 30, 32, 34, 36 or 43, when the sequence comparison is carried out over the entire length of the two sequences. In one or more additional embodiments, the amino acid sequence of said viral envelope protein comprises, consists essentially of, or consists of the amino acid sequence disclosed herein as SEQ ID NO:2, 4, 6, 8, 10, 12, 14, 16, 18, 20, 22, 24, 26, 28, 30, 32, 34, 36, or 43.

In another embodiment, the methods comprise a recombinant lentivirus comprising a viral envelope protein comprising at least one of the 31 amino acids within the CD34 cell transduction determinant shown in FIG. 4 at its respective location. In addition, the viral envelope protein comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or all 31 of the 31 amino acids within the CD34 cell transduction determinant shown in FIG. 4 at their respective locations.

In another embodiment, the method comprise a recombinant lentivirus comprising an arenavirus envelope protein. For example the arenavirus envelope protein originates from a Machupo, Junin, Ocozocoautla, Tacaribe, Tacaribe, Guanarito, Amapar, Cupixi, Sabia or Chapre virus. In addition, the methods comprise a recombinant lentivirus comprising a viral envelope protein comprising an amino acid sequence having at least 70%, 75%, 80%, 85%, 90%, 95%, 96%, 97%, 98% or 99% sequence identity to the amino acid sequence of SEQ ID NO:41, when the sequence comparison is carried out over the entire length of the two sequences. In one or more additional embodiments, the amino acid sequence of said viral envelope protein comprises, consists essentially of, or consists of the amino acid sequence disclosed herein as SEQ ID NO:41.

In any of the methods of the disclosure, the recombinant lentivirus comprises a vector; and wherein the vector comprises said heterologous transgene operably linked to a promoter. In addition, in any of the methods of the disclosure, the recombinant lentivirus of the disclosure comprises a self-activating (SIN) LTR.

In another embodiment, in any of the methods of the disclosure the hematopoietic stem cell is transduced with a heterologous transgene that encodes a human protein. Optionally, the heterologous transgene encodes a human hemoglobin protein.

In a further embodiment, any of the methods of the disclosure comprise a recombinant lentivirus comprising protein that is a ligand for binding to CD34+ cells. Optionally, the protein that is as a ligand for binding to CD34+ cells is present on the surface of said recombinant lentivirus. The protein that is as a ligand for binding to CD34+ cells comprises an amino acid sequence having at least 70%, 75%, 80%, 85%, 90%, 95%, 96%, 97%, 98% or 99% sequence identity to the amino acid sequence of SEQ ID NO:39, when the sequence comparison is carried out over the entire length of the two sequences. Optionally, the protein that is as a ligand for binding to human CD34+ cells comprises, consists essential of or consists of the amino acid sequence of SEQ ID NO:39.

In addition, any of the methods of the disclosure comprise a recombinant lentivirus that was produced by a cell having a concentration ratio of vector expressing the envelope protein and the vector expressing L-selectin ranging from 1:2 to 1:5. In addition, any of the methods of the disclosure comprise a recombinant lentivirus wherein the concentration ratio of the envelope protein and L-selectin ranges from 1:2 to 1:5.

In one embodiment, the transduction step of any of methods of the disclosure is performed on adherent hematopoietic stem cells. In another embodiment, the transduction step of any of methods of the disclosure is performed on hematopoietic stem cells in suspension.

In another aspect, the invention provides a recombinant lentivirus capable of transducing a hematopoietic stem cell, said recombinant lentivirus comprising a heterologous transgene and a viral envelope protein that originates from a species of vesiculovirus selected from the group consisting of Vesicular Stomatitis Virus G (VSV-G), Morreton, Maraba, Cocal, Alagoas and Carajas. For example, the invention provides for a recombinant lentivirus capable of transducing a hematopoietic stem cell, said recombinant lentivirus comprising a heterologous transgene and a viral envelope protein comprising at least one of the 31 amino acids within the CD34 cell transduction determinant shown in FIG. 4 at its respective location.

In an additional aspect, the invention provides for a recombinant lentivirus capable of transducing a hematopoietic stem cell, said recombinant lentivirus comprising a heterologous transgene and a viral envelope protein that originates from a species of arenavirus capable of using transferrin receptor type 1 (TfnR1) to infect cells. For example, the invention provides for a recombinant lentivirus wherein the arenavirus envelope protein originates from a Machupo virus.

In an aspect, the invention provides for a composition comprising any of the recombinant lentivirus of the disclosure and a pharmaceutically acceptable carrier.

In another aspect, the invention provides for methods of treating a hemoglobinopathic condition comprising administering a hematopoietic stem cell transduced with any of the recombinant lentivirus of the disclosure or any of the compositions of the disclosure. For example the hemoglobinopathic condition is sickle cell anemia or thalassemias.

In an additional aspect, the invention provides for use of a hematopoietic stem cell transduced with any of the recombinant lentivirus of the disclosure or any composition of the disclosure for the preparation of a medicament for the treatment of a hemoglobinopathic condition. For example the hemoglobinopathic condition is sickle cell anemia or thalassemias.

In addition, the invention provides for compositions comprising a hematopoietic stem cell transduced with any of the recombinant lentivirus of the disclosure for treating a hemoglobinopathic condition. For example the hemoglobinopathic condition is sickle cell anemia or thalassemias.

### BRIEF DESCRIPTION OF THE DRAWINGS

FIG. 1. Phylogenetic relationships, rhabdovirus subfamilies, and the percent (%) amino acid identity of rhabdovirus envelope proteins to the VSV Indiana envelope protein.

FIG. 2. Transduction of human CD34+ cells by lentiviruses produced using a pCCL GLOBE1 βAS3 genome and the indicated envelope protein.

FIG. 3. Vesiculovirus phylogeny. Vesiculovirus envelopes that have been tested for transduction of human CD34+ cells are shown. Those that are either old world or new world-derived are indicated. The new world-derived vesiculovirus envelopes that have higher or lower efficiencies of human CD34+ cell transduction are indicated.

FIG. 4A-FIG. 4C. Human CD34+ cell transduction determinant. The 3 vesiculovirus envelopes that poorly mediate transduction of human CD34+ cells (Isfahan (SEQ ID NO: 26), Piry (SEQ ID NO: 57), Chandipura (SEQ ID NO: 18)) cells and 8 vesiculovirus envelopes that can efficiently mediate transduction of human CD34+ cells (VSV-G (Arizona) (SEQ ID NO:4), VSV-G (Indiana) (SEQ ID NO:8), VSV-G (New Jersey) (SEQ ID NO: 14), Morreton (SEQ ID NO: 12), Maraba (SEQ ID NO: 10), Alagoas (SEQ ID NO: 2), Carajas (SEQ ID NO: 6), Cocal (SEQ ID NO: 43)) were aligned. A 31 amino acid human CD34+ cell transduction determinant that is found in all envelope proteins that can efficiently mediate transduction of human CD34+ cells but is not found in those that poorly mediate transduction of human CD34+ cells is shown.

FIG. 5. Location of amino acids in “human CD34+ cell transduction determinant” on the monomeric pre-fusion structure of VSV-G (Indiana). Amino acids that comprise the CD34+ cell transduction determinant are displayed in space filling mode while others are displayed in framework mode.

FIG. 6. Enhancement of lentiviral transduction of CD34+ cells by expression of human L-selectin in lentivirus producer cells.

FIG. 7. VSV-G Indiana mediated lentiviral transduction of CD34+ cells was not enhanced by co-expression of human SIGLEC10 in lentivirus producer cells, compared to L-selectin.

FIG. 8. Virus produced using 1 μg VSV-G Indiana plasmid and 5 μg of L-selectin plasmid (per 75 cm² flask) transduced CD34+ cells more efficiently than virus produced using 5 μg VSV-G Indiana plasmid.

FIG. 9. Effect of adding L-selectin expression vector (SELL) to optimized virus production containing 5 μg of VSV-G (Indiana) (IN) expression vector.

FIG. 10. Enhanced transduction of human CD34+ cells by lentiviruses produced with a VSV-G envelope protein from the Indiana strain of VSV-G in producer cells expressing human L-selectin is inhibited by an antibody that neutralizes human L-selectin.

FIG. 11. Transduction of CD34 negative cells (293T cells) by lentiviruses that express eGFP and were produced in the presence or absence of human L-selectin.

FIG. 12. Dose-relationship between Maraba envelope plasmid and L-Selectin plasmid expression during lentivirus production and its effect on lentivirus transduction of human CD34+ cells.

FIG. 13. Lentivirus pseudo-typed with Maraba envelope and L-selectin has enhanced transduction of human CD34+ cells from multiple donors compared to VSV-G (Indiana) envelope pseudo-typed lentivirus.

FIG. 14. Enhancement of Morreton vesiculovirus envelope-mediated transduction of human CD34+ cells by expression of human L-selectin in virus producing 293T cells.

FIG. 15. Enhancement of Carajas vessiculovirus envelope-mediate lentivirus transduction of human CD34+ cells by co-expression of human L-selectin in virus producing 293T cells

FIG. 16. Human CD34+ cells were transduced by lentiviruses produced with an arenavirus envelope protein from the Machupo virus (Carvallo strain).

FIG. 17. Phylogeny of arenavirus envelope proteins.

FIG. 18. Map of pHCMV-VSV-G (Indiana) (SEQ ID NO:44)

FIG. 19. Map of pHCMV-XL5-human L-Selectin (SEQ ID NO:45)

FIG. 20. Map of the eGFP reporter lentivirus genome plasmid (pCCL-c-MNU3-eGFP; SEQ ID NO:46).

FIG. 21. Map of pCCL GLOBEH3AS3 (SEQ ID NO:47)

FIG. 22. Map of pRSV rev (SEQ ID NO:48)

FIG. 23. Map of pMDL g/p RRE (SEQ ID NO:49)

### DETAILED DESCRIPTION OF THE INVENTION

The invention provides compositions and methods useful for producing lentiviruses with improved lentiviral transduction of hematopoietic stem cells required for gene therapy applications. The below described preferred embodiments illustrate adaptations of these compositions and methods. Nonetheless, from the description of these embodiments, other aspects of the invention can be made and/or practiced based on the description provided below.

### I. General Techniques

The practice of the present invention will employ, unless otherwise indicated, conventional techniques of cell biology, molecular biology, cell culture, virology, and the like which are in the skill of one in the art. These techniques are fully disclosed in current literature and reference is made specifically to Sambrook, Fritsch and Maniatis eds., “Molecular Cloning, A Laboratory Manual”, 2nd Ed., Cold Spring Harbor Laboratory Press (1989); Celis J. E. “Cell Biology, A Laboratory Handbook” Academic Press, Inc. (1994) and Bahnson et al., J. of Virol. Methods, 54:131-143 (1995). Furthermore, all publications and patent applications cited in this specification are indicative of the level of skill of those skilled in the art to which this invention pertains and are hereby incorporated by reference in their entirety.

### II. Definitions

Throughout the present disclosure, several terms are employed that are defined in the following paragraphs.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.”

As used herein, the term “about” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As used herein, the term “and/or” includes any and all combinations of one or more of the associated listed items.

The term “lentivirus” refers to a group of complex retroviruses, while the term “recombinant lentivirus” refers to a recombinant virus derived from lentivirus genome (such as an HIV-1 genome) engineered such that it cannot replicate but can be produced in cultured cells (e.g., 293T cells) and can deliver genes to cells of interest.

The term “vesiculovirus” refers to a genus of negative-sense single stranded retrovirus in the family of Rhabdoviridae.

The term “transduction” refers to the combined processes of infection of a cell of interest followed by gene delivery and expression.

The term “transduction determinant” refers to particular one or more amino acids within a viral envelope protein that mediate or enhance transduction of a cell by that virus. For example, a “CD34+ cell transduction determinant” refers to a set of amino acids found in a viral envelope protein that mediate or enhance transduction of CD34+ cells. These amino acids are used to pseudotype lentivirus, so that the resulting psuedotype lentivirus can transduce CD34+ cells to a similar or greater extent than the prototypical VSV-G Indiana pseudotyped lentivirus.

The term “envelope protein” refers to a transmembrane protein on the surface of a virus that determines what species and cell types the virus can transduce.

The term “pseudotyping” refers to the replacement of any component of a virus with that from a heterologous virus. In particular, “pseudotyping” denotes a recombinant virus comprising an envelope different from the wild-type envelope, and thus possessing a modified tropism. In the case of the pseudotyped lentiviruses, they are lentiviruses which have a heterologous envelope of non-lentiviral origin or a different species or subspecies of lentivirus, for example originating from another virus, or of cellular origin, or the envelope is replaced with another cellular membrane protein originating from another virus or cellular origin

The term “VSV envelope” refers to an envelope protein from a rhabdovirus called vesicular stomatitis virus (VSV). Often this protein is also referred to as the VSV-G protein where “G” means glycoprotein. The envelope protein of rhabdoviruses is the only rhabdovirus protein that is glycosylated.

The term “hematopoietic stem cell” refers to a cell, which when transplanted into a stem cell deficient recipient, can home to the bone marrow and divide and differentiate into terminally differentiated cells found in blood from the myeloid or erythroid lineages such as red blood cells, T cells, neutrophils, granulocytes, monocytes, natural killer cells, basophils, dendritic cells, eosinophils, mast cells, B cells, platelets, and megakaryocytes. In some embodiments the hematopoietic stem cell is a human hematopoietic stem cell.

CD34 is a glycosylated transmembrane protein which is commonly used as a marker for primitive blood- and bone marrow-derived progenitor cells, such as hematopoietic and endothelial stem cells. The term “CD34+ cell” refers to a cell which expresses the CD34 protein such as hematopoietic stem cells, endothelial stem cells and mesenchymal stem cells.

The term “adherent hematopoietic stem cells” refers to hematopoietic stem cells that attach to a solid or semi-solid substrate, such as the surface of a cell culture vessel or another suitable substrate. The adherent human hematopoietic stem cells will grow in vitro until they have covered the available surface area of the cell culture vessel or substrate or the medium is delete of nutrients.

The term “hematopoietic stem cells in suspension” refers to hematopoietic stem cells that grow in vitro but do not attach to the surface of a cell culture vessel and grow in vitro while floating in the culture medium.

The term “transgene” refers to an exogenous nucleic acid sequence that is introduced into a host cell or genome of an organism via a vector, such as a recombinant lentivirus vector. A “heterologous transgene” refers to an exogenous nucleic acid sequence from one organism that is introduced into a different organism that encodes a protein, peptide, polypeptide, enzyme, or another product of interest and regulatory sequences directing transcription and/or translation of the encoded product in a host cell, and which enable expression of the encoded product in the host cell. For example, a heterologous transgene is heterologous to the lentivirus sequences and enables expression of the encoded product in the host cell.

The term “sequence identity” refers to the similarity of two or more nucleotide or amino acid sequences, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program.

In order to determine the percent identity of two nucleic acid sequences, the sequences are aligned for optimal comparison purposes (e.g., gaps can be introduced in the sequence of a first nucleic acid for optimal alignment with a second amino or nucleic acid sequence). The nucleotide residues at nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are the same length.

A sequence comparison may be carried out over the entire lengths of the two sequences being compared or over fragment of the two sequences. Typically, the comparison will be carried out over the full length of the two sequences being compared. However, sequence identity may be carried out over a region of, for example, about twenty, about fifty, about one hundred, about two hundred, about five hundred, about 1000, about 2000, about 3000, about 4000, about 4500, about 5000 or more contiguous nucleic acid residues. Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

The term “ligand for binding to CD34+ cells” is a molecule that facilitates the lentivirus binding to the cell surface of the CD34+ cell for transduction. The ligand may be a protein, glycoprotein, sugar or lipid. An exemplary ligand for binding to human CD34+ cells is L-selectin. The term “vector” refers to a nucleic acid molecule that introduced a nucleic acid sequence into a cell. For example, a recombinant lentivirus serves as a vector for introducing a nucleic acid sequence into a human CD34+ cells.

The term “operably linked” refers to the association of nucleotide sequences on a single nucleic acid molecule, e.g. an expression cassette or a vector, in a way such that the function of one or more nucleotide sequences is affected by at least one other nucleotide sequence present on said nucleic acid molecule. For example, an expression control sequence, such as a promoter, is operably linked with a transgene, when it is capable of effecting the expression of that transgene nucleic acid sequence.

The term “promoter” refers to a nucleic acid sequence to which the enzyme RNA polymerase can bind to initiate the transcription of DNA into RNA. This is an expression control sequence that functions to facilitate expression of a transgene.

The term “self-inactivating lentivirus vector” refers to a lentivirus vector that contains a non-functional or modified 3′ Long Terminal Repeat (LTR) sequence. This sequence is copied to the 5′ end of the vector genome during integration, resulting in the inactivation of promoter activity by both LTRs.

### III. Description of Invention

A. Recombinant Lentivirus

The present invention provides recombinant viruses with lentiviral gene therapy vectors in combination with viral envelope proteins which enable transduction of hematopoietic stem cells, such as human CD34+ cells. In one embodiment, the invention provides a recombinant lentivirus composed of a lentivirus gene vector packaged in a heterologous envelope comprising the binding domain of a rhabdovirus envelope protein or an amino acid sequence derived therefrom. The lentiviral vector of the invention contains, at a minimum, lentivirus 5′ long terminal repeat (LTR) sequences. a molecule for delivery to the host cells, and a functional portion of the lentivirus 3′ LTR sequences. Optionally, the vector may further contain a ψ (psi) encapsidation sequence, Rev response element (RRE) sequences or sequences which provide equivalent or similar function. The heterologous molecule carried on the vector for delivery to a host cell may be any desired substance including, without limitation, a polypeptide, protein, enzyme, carbohydrate, chemical moiety, or nucleic acid molecule which may include oligonucleotides, RNA, DNA, and/or RNA/DNA hybrids. In one embodiment, the heterologous molecule is a nucleic acid molecule which introduces specific genetic modifications into human chromosomes, e.g., for correction of mutated genes. In another desirable embodiment, the heterologous molecule comprises a transgene comprising a nucleic acid sequence encoding a desired protein, peptide, polypeptide, enzyme, or another product and regulatory sequences directing transcription and/or translation of the encoded product in a host cell, and which enable expression of the encoded product in the host cell. Suitable products and regulatory sequences are discussed in more detail below. However, the selection of the heterologous molecule carried on the vector and delivered by the viruses of the invention is not a limitation of the present invention.

1. Lentiviral Elements

In selecting the lentiviral elements described herein for construction of the lentivirus vector and the recombinant virus of the invention, one may readily select sequences from any suitable lentivirus and any suitable lentivirus serotype or strain. Suitable lentiviruses include, for example, human immunodeficiency virus (HIV), simian immunodeficiency virus (SIV), caprine arthritis and encephalitis virus (CAEV), equine infectious anemia virus (EIAV), visna virus, and feline immunodeficiency virus (FIV), bovine immune deficiency virus (BIV). The examples provided herein illustrate the use of a vector derived from HIV. However, FIV and other lentiviruses of non-human origin may also be particularly desirable. The sequences used in the constructs of the invention may be derived from academic, non-profit (e.g., the American Type Culture Collection, Manassas, Va.) or commercial sources of lentiviruses. Alternatively, the sequences may be produced recombinantly, using genetic engineering techniques, or synthesized using conventional techniques (e.g., G. Barony and R. B. Merrifield, THE PEPTIDES: ANALYSIS, SYNTHESIS & BIOLOGY, Academic Press, pp. 3-285 (1980)) with reference to published viral sequences, including sequences contained in publicly accessible electronic databases.

a) LTR Sequences

The lentiviral vector contains a sufficient amount of lentiviral long terminal repeat (LTR) sequences to permit reverse transcription of the genome, to generate cDNA, and to permit expression of the RNA sequences present in the lentiviral vector. Suitably, these sequences include both the 5′ LTR sequences, which are located at the extreme 5′ end of the vector and the 3′ LTR sequences, which are located at the extreme 3′ end of the vector. These LTR sequences may be intact LTRs native to a selected lentivirus or a cross-reactive lentivirus, or more desirably, may be modified LTRs.

Various modifications to lentivirus LTRs have been described. One particularly desirable modification is a self-inactivating LTR, such as that described in H. Miyoshi et al, J. Virol., 72:8150-8157 (Oct. 1998) for HIV. In these HIV LTRs, the U3 region of the 5′ LTR is replaced with a strong heterologous promoter (e.g., CMV) and a deletion of 133 bp is made in the U3 region of the 3′ LTR. Thus, upon reverse transcription, the deletion of the 3′ LTR is transferred to the 5′ LTR, resulting in transcriptional inactivation of the LTR. The complete nucleotide sequence of HIV is known, see, L. Ratner et al. Nature. 313(6000):277-284 (1985). Yet another suitable modification involves a complete deletion in the U3 region, so that the 5′ LTR contains only a strong heterologous promoter, the R region, and the U5 region; and the 3′ LTR contains only the R region, which includes a polyA. In yet another embodiment, both the U3 and U5 regions of the 5′ LTRs are deleted and the 3′ LTRs contain only the R region. These and other suitable modifications may be readily engineered by one of skill in the art, in HIV and/or in comparable regions of another selected lentivirus.

Optionally, the lentiviral vector may contain a w (psi) packaging signal sequence downstream of the 5′ lentivirus LTR sequences. Optionally, one or more splice donor sites may be located between the LTR sequences and immediately upstream of the w sequence. According to the present invention, the w sequences may be modified to remove the overlap with the gag sequences and to improve packaging. For example, a stop codon may be inserted upstream of the gag coding sequence. Other suitable modifications to the w sequences may be engineered by one of skill in the art. Such modifications are not a limitation of the present invention.

In one suitable embodiment, the lentiviral vector contains lentiviral Rev responsive element (RRE) sequences located downstream of the LTR and w sequences. Suitably, the RRE sequences contain a minimum of about 275 to about 300 nt of the native lentiviral RRE sequences, and more preferably, at least about 400 to about 450 nt of the RRE sequences. Optionally, the RRE sequences may be substituted by another suitable element which assists in expression of gag/pol and its transportation to the cell nucleus. For example, other suitable sequences may include the CT element of the Manson-Pfizer virus, or the woodchuck hepatitis virus post-regulatory element (WPRE). Alternatively, the sequences encoding gag and gag/pol may be altered such that nuclear localization is modified without altering the amino acid sequences of the gag and gag/pol polypeptides. Suitable methods will be readily apparent to one of skill in the art.

b) Transgene

As stated above, in one desirable embodiment, the molecule carried by the lentiviral vector is a transgene. The transgene is a nucleic acid molecule comprising a nucleic acid sequence, heterologous to the lentiviral sequences, which encodes a protein, peptide, polypeptide, enzyme, or another product of interest and regulatory sequences directing transcription and/or translation of the encoded product in a host cell, and which enable expression of the encoded product in the host cell. The composition of the transgene depends upon the intended use for the vector and the pseudotyped virus of the invention.

For example, one type of transgene comprises a reporter or marker sequence which, upon expression, produces a detectable signal. Such reporter or marker sequences include, without limitation, DNA sequences encoding β-lactamase, β-galactosidase (LacZ). alkaline phosphatase, thymidine kinase, green fluorescent protein (GFP), chloramphenicol acetyltransferase (CAT), luciferase, membrane bound proteins including, for example, CD2, CD4, CD8, and the influenza hemagglutinin protein, as well as others well known in the art. In an alternative, the recombinant viruses of the invention are useful for delivery of gene products and other molecules which induce an antibody and/or cell-mediated immune response, e.g., for vaccine purposes. Suitable gene products may be readily selected by one of skill in the art from among immunogenic proteins and polypeptides derived from viruses, as well as from prokaryotic and eukaryotic organisms, including unicellular and multicellular parasites. In another alternative, the recombinant viruses of the invention are useful for delivery of a molecule desirable for study.

In one particularly desirable embodiment, the recombinant viruses of the invention are useful for therapeutic purposes, including, without limitation, correcting or ameliorating gene deficiencies, wherein normal genes are expressed but at less than normal levels. The recombinant viruses may also be used to correct or ameliorate genetic defects wherein a functional gene product is not expressed. A preferred type of transgene contains a sequence encoding a desired therapeutic product for expression in a host cell. These therapeutic nucleic acid sequences typically encode products which, upon expression, are able to correct or complement an inherited or non-inherited genetic defect, or treat an epigenetic disorder or disease. Thus, the invention includes methods of producing a recombinant virus which can be used to correct or ameliorate a gene defect caused by a multi-subunit protein. In certain situations, a different transgene may be used to encode each subunit of the protein. This is desirable when the size of the DNA encoding the protein subunit is large, e.g., for an immunoglobulin or the platelet-derived growth factor receptor. In order for the cell to produce the multi-subunit protein, a cell would be infected with recombinant viruses containing each of the different subunits. Alternatively, different subunits of a protein may be encoded by the same transgene. In this case, a single transgene would include the DNA encoding each of the subunits, with the DNA for each subunit separated by an internal ribosome entry site (IRES). This is desirable when the size of the DNA encoding each of the subunits is small, such that the total of the DNA encoding the subunits and the IRES is less than nine kilobases. Alternatively, other methods which do not require the use of an IRES may be used for co-expression of proteins. Such other methods may involve the use of a second internal promoter, an alternative splice signal, or a co- or post-translational proteolytic cleavage strategy, among others which are known to those of skill in the art. In one particular embodiment of the invention the gene product encoded by the transgene is functional human hemoglobin protein.

Other useful transgenes include non-naturally occurring polypeptides. such as chimeric or hybrid polypeptides or polypeptides having a non-naturally occurring amino acid sequence containing insertions, deletions or amino acid substitutions. Other types of non-naturally occurring gene sequences include antisense molecules and catalytic nucleic acids, such as ribozymes, which could be used to reduce overexpression of a gene. The selection of the transgene sequence, or other molecule carried by the lentiviral vector, is not a limitation of this invention. Choice of a transgene sequence is within the skill of the artisan in accordance with the teachings of this application.

c) Regulatory Elements

Design of a transgene or another nucleic acid sequence that requires transcription, translation and/or expression to obtain the desired gene product in cells and hosts may include appropriate sequences that are operably linked to the coding sequences of interest to promote expression of the encoded product. “Operably linked” sequences include both expression control sequences that are contiguous with the nucleic acid sequences of interest and expression control sequences that act in trans or at a distance to control the nucleic acid sequences of interest.

Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. A great number of expression control sequences ˜ native, constitutive, inducible and/or tissue-specific—are known in the art and may be utilized to drive expression of the gene, depending upon the type of expression desired. For eukaryotic cells, expression control sequences typically include a promoter, an enhancer, such as one derived from an immunoglobulin gene, SV40, cytomegalovirus, etc. and a polyadenylation sequence which may include splice donor and acceptor sites. The polyadenylation (polyA) sequence generally is inserted following the transgene sequences and before the 3′ lentivirus LTR sequence. Most suitably, the lentiviral vector carrying the transgene or other molecule contains the polyA from the lentivirus providing the LTR sequences, e.g., HIV. However, other source of polyA may be readily selected for inclusion in the construct of the invention. In one embodiment, the bovine growth hormone polyA is selected. A lentiviral vector of the present invention may also contain an intron, desirably located between the promoter/enhancer sequence and the transgene. One possible intron sequence is also derived from SV-40, and is referred to as the SV-40 T intron sequence. Another element that may be used in the vector is an internal ribosome entry site (IRES). An IRES sequence is used to produce more than one polypeptide from a single gene transcript. An IRES sequence would be used to produce a protein that contains more than one polypeptide chain. Selection of these and other common vector elements are conventional and many such sequences are available (see, e.g., Sambrook et al. and references cited therein at, for example, pages 3.18-3.26 and 16.17-16.27 and Ausubel et al., CURRENT PROTOCOLS IN MOLECULAR BIOLOGY. John Wiley & Sons, New York, 1989).

In one embodiment, high-level constitutive expression will be desired. Examples of useful constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EFlα promoter (Invitrogen). Inducible promoters, regulated by exogenously supplied compounds, are also useful and include, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA. 93:3346-3351 (1996)), the tetracycline-repressible system (Gossen et al. Proc. Natl. Acad. Sci. USA, 89:5547-5551 (1992)), the tetracycline-inducible system (Gossen et al, Science. 268: 1766-1769 (1995), see also Harvey et al, Curr. Opin. Chem. Biol. 2:512-518 (1998)), the RU486-inducible system (Wang et al. Nat. Biotech. 15:239-243 (1997) and Wang et al, Gene Ther. 4:432-441 (1997)) and the rapamycin-inducible system (Magari et al, J. Clin. Invest. 100:2865-2872 (1997)). Other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally or in a tissue-specific manner, or in response to specific transcriptional stimuli. In a further embodiment, other native expression control elements, such as enhancer elements, polyadenylation sites or Kozak consensus sequences may also be used to mimic the native expression. Another embodiment of the transgene includes a transgene operably linked to a tissue-specific promoter.

Not all expression control sequences will function equally well to express all of the transgenes of this invention. However, one of skill in the art may make a selection among these expression control sequences without departing from the scope of this invention. Suitable promoter/enhancer sequences may be selected by one of skill in the art using the guidance provided by this application. Such selection is a routine matter and is not a limitation of the molecule or construct. For instance, one may select one or more expression control sequences may be operably linked to the coding sequence of interest, and inserted into the transgene, the vector, and the recombinant virus of the invention. After following one of the methods for packaging the lentivirus vector taught in this specification, or as taught in the art, one may infect suitable cells in vitro or in vivo. The number of copies of the vector in the cell may be monitored by Southern blotting or quantitative PCR. The level of RNA expression may be monitored by Northern blotting or quantitative RT-PCR. The level of expression may be monitored by Western blotting, immunohistochemistry, ELISA, RIA or tests of the gene product's biological activity. Thus, one may easily assay whether a particular expression control sequence is suitable for a specific produced encoded by the transgene, and choose the expression control sequence most appropriate. Alternatively, where the molecule for delivery does not require expression, e.g., a carbohydrate, polypeptide, peptide, etc., the expression control sequences need not form part of the lentiviral vector or other molecule.

d) Other Lentiviral Elements

Optionally, the lentivirus vector may contain other lentiviral elements, such as those well known in the art, many of which are described below in connection with the lentiviral packaging sequences. However, notably, the lentivirus vector lacks the ability to assemble lentiviral envelope protein. Such a lentivirus vector may contain a portion of the envelope sequences corresponding to the RRE but lack the other envelope sequences. However, more desirably, the lentivirus vector lacks the sequences encoding any functional lentiviral envelope protein in order to substantially eliminate the possibility of a recombination event which results in replication competent virus.

Thus, the lentiviral vector of the invention contains, at a minimum, lentivirus 5′ long terminal repeat (LTR) sequences, (optionally) a w (psi) encapsidation sequence, a molecule for delivery to the host cells, and a functional portion of the lentivirus 3′ LTR sequences. Desirably, the vector further contains RRE sequences or their functional equivalent. Suitably, a lentiviral vector of the invention is delivered to a host cell for packaging into a virus by any suitable means, e.g., by transfection of the “naked” DNA molecule comprising the lentiviral vector or by a vector which may contain other lentiviral and regulatory elements described above, as well as any other elements commonly found on vectors. A “vector” can be any suitable vehicle which is capable of delivering the sequences or molecules carried thereon to a cell. For example, the vector may be readily selected from among, without limitation, a plasmid, phage, transposon, cosmid, virus, etc. Plasmids are particularly desirable for use in the invention. The selected vector may be delivered by any suitable method, including transfection, electroporation, liposome delivery, membrane fusion techniques, high velocity DNA-coated pellets, viral infection and protoplast fusion. According to the present invention, the lentiviral vector is packaged in a heterologous (i.e., non-lentiviral) envelope using the methods described in part B below to form the recombinant virus of the invention.

2. Envelope Protein

The envelope in which the lentiviral vector is packaged is suitably free of lentiviral envelope protein and comprises the binding domain of at least one heterologous envelope protein. In one embodiment, the envelope may be derived entirely from rhabdovirus glycoprotein or may contain a fragment of the rhabdovirus envelope (a rhabdovirus polypeptide or peptide) which contains the binding domain fused in frame to an envelope protein, polypeptide, or peptide, of a second virus. In an alternative, the envelope may contain a viral envelope protein comprising a sequence derived from the CD34+ cell transduction determinant shown in FIG. 4 and discussed below. In another embodiment, the envelope may be derived entirely from arenavirus glycoprotein or a fragment thereof.

a) Rhabdovirus Envelope Proteins

The rhabdovirus which provides the sequences encoding the envelope protein or a polypeptide or peptide thereof (e.g., the binding domain) can be derived from any suitable serotype from the vesiculovirus subfamily, e.g. VSV-G (Indiana), Morreton, Maraba, Cocal, Alagoa, Carajas, VSV-G (Arizona), Isfahan, VSV-G (New Jersey), or Piry. The sequences encoding the envelope protein may be obtained by any suitable means, including application of genetic engineering techniques to a viral source, chemical synthesis techniques, recombinant production or combinations thereof. Suitable sources of the desired viral sequences are well known in the art, and include a variety of academic, non-profit, commercial sources, and from electronic databases. The methods by which the sequences are obtained is not a limitation of the present invention. In one desirable embodiment, the heterologous envelope sequences are derived from a 31 amino acid human CD34+ cell transduction determinant that is found in all envelope proteins that can mediate transduction of human CD34+ cells but is not found in those that do not mediate transduction of human CD34+ cells.

Thus, in one embodiment, the envelope protein is intact rhabdovirus glycoprotein. Alternatively, it may be desirable to utilize a fragment of the selected rhabdovirus which contains, at a minimum, the binding domain of the rhabdovirus envelope glycoprotein, which is located within a 31 amino acid human CD34+ cell transduction determinant. Suitably, this rhabdovirus protein fragment is fused, directly or indirectly, via a linker, to a second, non-lentiviral, envelope protein or fragment thereof. This fusion protein may be desirable to improve packaging, yield, and/or purification of the resulting envelope protein. The second, non-lentiviral envelope protein or fragment thereof contains, at a minimum, the membrane domain. In one desirable embodiment, a truncated fragment of the 31 amino acid human CD34+ cell transduction determinant is fused to a VSV-G envelope protein. Still other fusion (chimeric) proteins according to the present invention can be generated by one of skill in the art.

b) Arenavirus Envelope Proteins

In another embodiment, the envelope protein is an intact arenavirus envelope protein or a fragment of the selected arenavirus envelope protein which contains, at a minimum, the binding domain of the arenavirus envelope glycoprotein. Suitably, this arenavirus protein fragment is fused, directly or indirectly, via a linker, to a second, non-lentiviral, envelope protein or fragment thereof. This fusion protein may be desirable to improve packaging, yield, and/or purification of the resulting envelope protein. The second, non-lentiviral envelope protein or fragment thereof contains, at a minimum, the membrane domain.

Protective neutralizing antibody immunity against the arenaviral envelope glycoprotein (GP) is minimal, meaning that infection results in minimal antibody-mediated protection against re-infection if any. This characteristic allows for repeated immunization with vectors comprising the arenavirus envelope protein. Pre-existing immunity for arenavirus is low or negligible in the human population. In addition, arenavirus are generally non-cytolytic (not cell-destroying), and may under certain conditions, maintain long-term antigen expression in animals without eliciting disease.

Arenavirus envelope proteins may be from Lassa virus. Luna virus, Lujo virus, Lymphocytic choriomeningitis virus (LCMV), Mobala virus, Mopeia virus, Ippy virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, Whitewater Arroyo virus, Merino walk virus, Menekre virus, Morogoro virus, Gbagroube virus, Kodoko virus, Lemniscomys virus, Mus minutoides virus, Lunk virus, Giaro virus, and Wenzhou virus, Patawa virus, Pampa virus, Tonto Creek virus, Allpahuayo virus, Catarina virus, Skinner Tank virus, Real de Catorce virus, Big Brushy Tank virus, Catarina virus, and Ocozocoautla de Espinosa virus.

c) Chimeric Envelope Glycoproteins

In another embodiment, a useful envelope may be a chimeric glycoprotein containing the binding domain of a rhabdovirus or arenavirus envelope glycoprotein fused to a fragment of a second envelope glycoprotein or a non-contiguous fragment of a rhabdovirus or arenavirus capsid protein. For example, a selected rhabdovirus or arenavirus binding domain may be fused to a transmembrane domain of the same or another selected rhabdovirus or arenavirus strain. In another embodiment, the second protein or fragment may be derived from another non-lentiviral source. For example, one suitable envelope protein may contain the membrane domain from vesicular stomatitis virus (VSV) glycoprotein (G). Alternatively, other suitable fragment may be selected from another suitable viral source which provides the desired packaging levels. Where the envelope is a fusion protein, a linker may be inserted between the sequences encoding the rhabdovirus or arenavirus envelope protein (or fragment thereof) and the sequences encoding the second envelope protein (or fragment thereof). Such a linker may desirable, in order to ensure that, upon expression, an envelope which is a fusion protein is produced. Thus, the linker may be a spacer which ensures that the two sequences are appropriately translated. Such a linker may be nucleic acids (preferably non-coding sequences) or it may be a chemical compound or other suitable moiety. Suitable techniques for designing such a fusion protein are well known to those of skill in the art. See, generally, Sambrook et al, Molecular Cloning: A Laboratory Manual, Cold Spring Harbor Laboratories, Cold Spring Harbor. New York.

3. Ligand for Binding CD34+ Cells

The expression of a ligand for binding CD32+ cells in lentivirus producing cells resulted in the production of lentivirus exhibiting enhanced transduction of hematopoietic stem cells. CD34+ hematopoietic stem cells bind to ligands on the cell surface which facilitate the lentivirus binding to the cell surface for transduction. The ligand may be a protein, glycoprotein, sugar or lipid. A particular example of a CD34+ cell ligand is L-selectin. Selectins are lectins that bind to specialized carbohydrate determinants, consisting of sialofucosylations containing an α(2,3)-linked sialic acid substitution(s) and an α(1,3)-linked fucose modification(s) prototypically displayed as the tetrasaccharide sialyl Lewis X (sLe.sup.x; Neu5Ac.alpha.2-3Gal.beta.1-4[Fuc.alpha.1-3]GlcNAc.beta.1-)) (1, 6). L-selectin is expressed on circulating leukocytes and expression of L-selectin in lentivirus producing cells was shown to enhance lentivirus transduction of CD34+ hematopoietic stem cells.

### IV. Production of Recombinant Transfer Virus

The invention further involves a method of producing a recombinant virus useful for delivering a selected molecule to a host cell. To produce recombinant transfer virus, the lentivirus transfer virus construct, gag, pol, an envelope protein and rev into the same or multiple vectors.

The recombinant transfer virus is a retrovirus or lentivirus that is capable of providing efficient delivery, integration and long term expression of transgenes into non-dividing cells both in vitro and in vivo. A variety of lentiviral vectors are known in the art, see Naldini et al., (1996a, 1996b, and 1998); Zufferey et al., (1997); Dull et al., 1998, U.S. Pat. Nos. 6,013,516; and 5,994,136, any of which may be adapted to produce a transfer vector of the present invention. In general, these vectors are plasmid-based or virus-based, and are configured to carry the essential sequences for transfer of a nucleic acid encoding a therapeutic polypeptide into a host cell.

A. Methods of Producing Recombinant Lentivirus

The recombinant lentivirus is replication defective, and therefore the virus is produced in a “producer cell line” in which the necessary constituents are provided in a single cell. As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

Three or four separate plasmid systems are used to generate the producer cell line. The four plasmid system comprises three helper plasmids and one transfer vector plasmid. For example, the Gag-Pol expression cassette encodes structural proteins and enzymes. Another cassette encodes Rev, which is an accessory protein necessary for vector genome nuclear export. A third cassette encodes a heterologous envelope protein, such as a vesiculovirus or arenavirus envelope protein, that allows lentivirus particle entry into target cells. The transfer vector cassette encodes the vector genome itself, which carries signals for incorporation into particles and an internal promoter driving transgene expression. The transfer vector carries the heterologous transgene and is the only genetic material is transferred to the target cells, e.g. CD34+ cell. The three plasmid system comprises two helper plasmids coding for the gag-pol and the envelope functions and the transfer vector cassette. See Merten et al., Mol. Ther. Methods Clin. Dev. 3: 16017, 2016.

The multiple constituent expression cassettes are transiently or stably transfected in the producer cell. In one embodiment, the producer cell line in which the necessary constituents are continuously and constitutively produced. The producer cell may be HEK293 cells, HEK293T cells,2 93FT, 293SF-3F6, SODk1 cells, CV-1 cells, COS-1 cells, HtTA-1 cells, STAR cells, RD-MolPack cells, Win-Pac, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, B-50 cells, 3T3 cells, NIH3T3 cells, HepG2 cells, Saos-2 cells, Huh? cells, HeLa cells, W163 cells, 211 cells, and 211A cells. There are commercially available lentivirus packaging systems, e.g. LentiSuite Kit (Systems Biosciences, Palo Alto, Calif.), Lenti-X packaging system (Takara Bio, Mountain View, Calif.), ViraSafe Packaging System (Cell Biolabs, Inc. San Diego, Calif.), ViroPower Lentiviarl Packaging Mix (Invitrogen) and Mission Lentiviral Packaging mix (Millapore Sigma, Burlington, Mass.).

In another embodiment, producer cell lines comprise inducible expression cassettes to express the packaging function. For example, the tetracycline-inducible expression system is used to generate the producer cells including the TET-Off system and the TET-On system. In addition, the ecdysone-inducible system is used.

Lentivirus production is performed using surface adherent cells grown in Petri dishes, T-flasks, multitray systems (Cell Factories, Cell Stacks), or HYPERFlask. At optimal confluence (<50%), cells are transfected using either the traditional Ca-phosphate protocol or the more recently developed polyethylenimine (PEI) method. Other efficient cationic transfection agents that are used include lipofectamine (Thermo-Fisher), fugene (Promega) LV-MAX (Thermo-Fisher), TransIT (Mirus) or 293fectin (Thermo-Fisher).

Alternatively, lentivirus production is performed using suspension cultures using shaker flasks, glass bioreactors, stainless steel bioreactor, wave bags, and disposable stirred tanks. The suspension cultures are transfected using Ca-phosphate or cationic polymers, and linear polyethyleneimine. The cells are also transfected using electroporation.

Purification of the lentivirus is carried out using membrane process steps such as filtration/clarification, concentration/diafiltration using tangential flow filtration (TFF) or membrane-based chromatography, and/or chromatography process steps such as ion-exchange chromatography (IEX), affinity chromatography, and size exclusion chromatography-based process steps. Any combination of these processes are used to purify the lentivirus. A benzonase/DNase treatment for the degradation of contaminating DNA is either part of the downstream protocol or is performed during vector production.

Purification is carried out three phases: (i) capture is the initial purification of the target molecule from either crude or clarified cell culture and leads to elimination of major contaminants. (ii) intermediate purification consists of steps performed on clarified feed between capture and polishing stages which results in removing specific impurities (proteins, DNA, and endotoxins), (iii) polishing is the final step aiming at removing trace contaminants and impurities leaving an active and safe product in a form suitable for formulation or packaging. Contaminants are often conformer to the target molecule, trace amounts of other impurities or suspected leakage products. Any type of chromatography and ultrafiltration process are used for the intermediate purification and the final polishing step(s).

Exemplary standard processes for purification of lentivirus include i) for removal of removal of cells and debris carried out with frontal filtration (0.45 μm) or centrifugation, ii) capture chromatography is carried out with anion-exchange chromatography such as Mustang Q or DEAE Sepharose, or affinity chromatography (heparin), iii) polishing is carried out with size-exclusion chromatography, iv) concentration and buffer exchange is carried out with tangential flow filtration or ultracentrifugation, v) DNA reduction is carried out with Benzonase and vi) sterilization is carried out with a 0.2-μm filter. See Merten et al., Mol. Ther Methods Clin Dev. 3: 16017, 2016.

B. Methods of Enhancing Transduction of Target Cells

At the initiation of transduction, the binding of virus particles to target cell is mediated by specific interactions between the viral envelope and specific receptors on the cell surface. However, several recent studies have demonstrated that the initial step of virus binding does not involve specific envelope-receptor interactions but rather receptor-independent binding events (Pizzato M, Marlow S A, Blair E D, Takeuchi Y. Initial binding of murine leukemia virus particles to cells does not require specific Env-receptor interaction. J. Virol. 1999; 73(10):8599-8611; Sharma S, Miyanohara A, Friedmann T. Separable mechanisms of attachment and cell uptake during retrovirus infection. J. Virol. 2000; 74(22):10790-10795). The efficiency of this initial event and, consequently, lentiviral transduction is diminished by strong electrostatic repulsion between the negatively charged cell and an approaching enveloped virus (Jensen T W, Chen Y, Miller W M. Small increases in pH enhance retroviral vector transduction efficiency of NIH-3T3 cells. Biotechnol. Prog. 2003; 19(1):216-223; Swaney W P, Sorgi F L, Bahnson A B, Barranger J A. The effect of cationic liposome pretreatment and centrifugation on retrovirus-mediated gene transfer. Gene Ther. 1997; 4(12):1379-1386). Methods designed to overcome this problem include centrifugation of targets cells with virus at low speeds, co-localization of cells and virus on immobilized proteins, and employing multiple rounds of transduction (Swaney et al. supra; O'Doherty U, Swiggard W J, Malim M H. Human immunodeficiency virus type 1 spinoculation enhances infection through virus binding. J. Virol. 2000; 74(21):10074-10080). Importantly, the addition of positively-charged polycations such as polybrene, DEAE-dextran, protamine sulfate, poly-L-lysine, or cationic liposomes reduces the repulsion forces between the cell and the virus and mediates the binding of retroviral particle to the cell surface resulting in a higher efficiency of transduction (Swaney et al. supra; Toyoshima K, Vogt P K. Enhancement and inhibition of avian sarcoma viruses by polycations and polyanions. Virology. 1969; 38(3):414-426; Le Doux J M, Landazuri N, Yarmush M L, Morgan J R. Complexation of retrovirus with cationic and anionic polymers increases the efficiency of gene transfer. Hum. Gene Ther. 2001; 12(13):1611-1621; Hodgson C P, Solaiman F. Virosomes: cationic liposomes enhance retroviral transduction. Nat. Biotechnol. 1996; 14(3):339-342; Cornetta K, Anderson W F. Enhanced in vitro and in vivo gene delivery using cationic agent complexed retrovirus vectors. Gene Ther. 1998; 5(9):1180-1186; Seitz B, Baktanian E, Gordon E M, Anderson W F, LaBree L, McDonnell P J.

C. Pharmaceutical Compositions and Formulations

The invention provides for pharmaceutical compositions and formulations comprising lentivirus transduced cells, such as hematopoietic stem cells and more specifically CD34+ cells, produced according to methods described herein and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media.

In one embodiment, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the transduced cells, use thereof in the pharmaceutical compositions of the invention is contemplated.

The compositions of the invention are administered alone or in combination with other agents as well, such as, e.g., cytokines, growth factors, hormones, small molecules or various pharmaceutically-active agents. There is virtually no limit to other components that may also be included in the compositions, provided that the additional agents do not adversely affect the ability of the composition to deliver the intended gene therapy.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

In certain circumstances it will be desirable to deliver the compositions disclosed herein parenterally, intravenously, intramuscularly, or even intraperitoneally as described, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, optionally mixing with CPP polypeptides, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques. The formulations and compositions of the invention may comprise one or more repressors and/or activators comprised of a combination of any number of polypeptides, polynucleotides, and small molecules, as described herein, formulated in pharmaceutically-acceptable or physiologically-acceptable solutions (e.g., culture medium) for administration to a cell or an animal, either alone, or in combination with one or more other modalities of therapy. It will also be understood that, if desired, the compositions of the invention may be administered in combination with other agents as well, such as, e.g., cells, other proteins or polypeptides or various pharmaceutically-active agents.

In a particular embodiment, a formulation or composition according to the present invention comprises a cell contacted with a combination of any number of polypeptides, polynucleotides, and small molecules, as described herein.

In certain aspects, the present invention provides formulations or compositions suitable for the delivery of viral vector systems (i.e., viral-mediated transduction) including, but not limited to, retroviral (e.g., lentiviral) vectors.

Exemplary formulations for ex vivo delivery may also include the use of various transfection agents known in the art, such as calcium phosphate, electoporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In certain aspects, the present invention provides pharmaceutically acceptable compositions which comprise a therapeutically-effective amount of one or more polynucleotides or polypeptides, as described herein, formulated together with one or more pharmaceutically acceptable carriers (additives) and/or diluents (e.g., pharmaceutically acceptable cell culture medium).

Particular embodiments of the invention may comprise other formulations, such as those that are well known in the pharmaceutical art, and are described, for example, in Remington: The Science and Practice of Pharmacy, 20th Edition. Baltimore, Md.: Lippincott Williams & Wilkins, 2000.

D. Methods of Treatment

The recombinant lentivirus provide improved methods of gene therapy. As used herein, the term “gene therapy” refers to the introduction of a polynucleotide into a cell's genome that restores, corrects, or modifies the gene and/or expression of the gene. In various embodiments, a viral vector of the invention comprises a hematopoietic expression control sequence that expresses a therapeutic transgene encoding a polypeptide that provides curative, preventative, or ameliorative benefits to a subject diagnosed with or that is suspected of having monogenic disease, disorder, or condition or a disease, disorder, or condition of the hematopoietic system. In addition, vectors of the invention comprise another expression control sequence that expresses a truncated erythropoietin receptor in a cell, in order to increase or expand a specific population or lineage of cells, e.g., erythroid cells. The virus can infect and transduce the cell in vivo, ex vivo, or in vitro. In ex vivo and in vitro embodiments, the transduced cells can then be administered to a subject in need of therapy. The present invention contemplates that the vector systems, viral particles, and transduced cells of the invention are be used to treat, prevent, and/or ameliorate a monogenic disease, disorder, or condition or a disease, disorder, or condition of the hematopoietic system in a subject, e.g., a hemoglobinopathy.

As used herein, “hematopoiesis,” refers to the formation and development of blood cells from progenitor cells as well as formation of progenitor cells from stem cells. Blood cells include but are not limited to erythrocytes or red blood cells (RBCs), reticulocytes, monocytes, neutrophils, megakaryocytes, eosinophils, basophils, B-cells, macrophages, granulocytes, mast cells, thrombocytes, and leukocytes.

As used herein, the term “hemoglobinopathy” or “hemoglobinopathic condition” includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease).

The term “sickle cell anemia” or “sickle cell disease” is defined herein to include any symptomatic anemic condition which results from sickling of red blood cells. Manifestations of sickle cell disease include: anemia; pain; and/or organ dysfunction, such as renal failure, retinopathy, acute-chest syndrome, ischemia, priapism and stroke. As used herein the term “sickle cell disease” refers to a variety of clinical problems attendant upon sickle cell anemia, especially in those subjects who are homozygotes for the sickle cell substitution in HbS. Among the constitutional manifestations referred to herein by use of the term of sickle cell disease are delay of growth and development, an increased tendency to develop serious infections, particularly due to pneumococcus, marked impairment of splenic function, preventing effective clearance of circulating bacteria, with recurrent infarcts and eventual destruction of splenic tissue. Also included in the term “sickle cell disease” are acute episodes of musculoskeletal pain, which affect primarily the lumbar spine, abdomen, and femoral shaft, and which are similar in mechanism and in severity to the bends. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia and dehydration, all of which potentiate intracellular polymerization of HbS (J. H. Jandl, Blood: Textbook of Hematology, 2nd Ed., Little, Brown and Company, Boston, 1996, pages 544-545). As used herein, the term “thalassemia” encompasses hereditary anemias that occur due to mutations affecting the synthesis of hemoglobin. Thus, the term includes any symptomatic anemia resulting from thalassemic conditions such as severe or β-thalassemia, thalassemia major, thalassemia intermedia, α-thalassemias such as hemoglobin H disease.

As used herein, “thalassemia” refers to a hereditary disorder characterized by defective production of hemoglobin. Examples of thalassemias include α and β thalassemia. β-thalassemias are caused by a mutation in the beta globin chain, and can occur in a major or minor form. In the major form of β-thalassemia, children are normal at birth, but develop anemia during the first year of life. The mild form of β-thalassemia produces small red blood cells.

α-thalassemias are caused by deletion of a gene or genes from the globin chain. α thalassemia typically results from deletions involving the HBA1 and HBA2 genes. Both of these genes encode an α-globin, which is a component (subunit) of hemoglobin. There are two copies of the HBA1 gene and two copies of the HBA2 gene in each cellular genome. As a result, there are four alleles that produce α-globin. The different types of α-thalassemia result from the loss of some or all of these alleles. Hb Bart syndrome, the most severe form of α-thalassemia, results from the loss of all four α-globin alleles. HbH disease is caused by a loss of three of the four α-globin alleles. In these two conditions, a shortage of α-globin prevents cells from making normal hemoglobin. Instead, cells produce abnormal forms of hemoglobin called hemoglobin Bart (Hb Bart) or hemoglobin H (HbH). These abnormal hemoglobin molecules cannot effectively carry oxygen to the body's tissues. The substitution of Hb Bart or HbH for normal hemoglobin causes anemia and the other serious health problems associated with α-thalassemia.

In a particular embodiment, gene therapy methods of the invention are used to treat, prevent, or ameliorate a hemoglobinopathy selected from the group consisting of: hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease.

In various embodiments, the lentivirus vectors are administered by direct injection to a cell, tissue, or organ of a subject in need of gene therapy, in vivo. In various other embodiments, cells are transduced in vitro or ex vivo with vectors of the invention, and optionally expanded ex vivo. The transduced cells are then administered to a subject in need of gene therapy.

In various embodiments, the use of hematopoietic stem cells for the gene therapy methods is preferred because they have the ability to differentiate into the appropriate cell types when administered to a particular biological niche, in vivo. The term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent. “Self-renewal” refers a cell with a unique capacity to produce unaltered daughter cells and to generate specialized cell types (potency). Self-renewal can be achieved in two ways. Asymmetric cell division produces one daughter cell that is identical to the parental cell and one daughter cell that is different from the parental cell and is a progenitor or differentiated cell. Asymmetric cell division does not increase the number of cells. Symmetric cell division produces two identical daughter cells. “Proliferation” or “expansion” of cells refers to symmetrically dividing cells.

As used herein, the term “pluripotent” means the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. As used herein, the term “multipotent” refers to the ability of an adult stem cell to form multiple cell types of one lineage. For example, hematopoietic stem cells are capable of forming all cells of the blood cell lineage, e.g., lymphoid and myeloid cells.

As used herein, the term “progenitor” or “progenitor cells” refers to cells that have the capacity to self-renew and to differentiate into more mature cells. Progenitor cells have a reduced potency compared to pluripotent and multipotent stem cells. Many progenitor cells differentiate along a single lineage, but may also have quite extensive proliferative capacity.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). When transplanted into lethally irradiated animals or humans, hematopoietic stem and progenitor cells can repopulate the erythroid, neutrophil-macrophage, megakaryocyte and lymphoid hematopoietic cell pool.

In preferred embodiments, the transduced cells are hematopoietic stem and/or progenitor cells isolated from bone marrow, umbilical cord blood, or peripheral circulation. In particular preferred embodiments, the transduced cells are hematopoietic stem cells isolated from bone marrow, umbilical cord blood, or peripheral circulation.

HSCs may be identified according to certain phenotypic or genotypic markers. For example, HSCs may be identified by their small size, lack of lineage (lin) markers, low staining (side population) with vital dyes such as rhodamine 123 (rhodamineDULL, also called rholo) or Hoechst 33342, and presence of various antigenic markers on their surface, many of which belong to the cluster of differentiation series (e.g., CD34, CD38, CD90, CD133, CD105, CD45, Ter119, and c-kit, the receptor for stem cell factor). HSCs are mainly negative for the markers that are typically used to detect lineage commitment, and, thus, are often referred to as Lin(−) cells.

In one embodiment, human HSCs may be characterized as CD34+, CD59+, Thy1/CD90⁺ CD38⁻, C-kit/CD117^{k}, CD49f⁺ and Lin(−). However, not all stem cells are covered by these combinations, as certain HSCs are CD34⁻/CD38⁻. Also some studies suggest that earliest stem cells may lack c-kit on the cell surface. For human HSCs, CD133 may represent an early marker, as both CD34+ and CD34− HSCs have been shown to be CD133+. It is known in the art that CD34+ and Lin(−) cells also include hematopoietic progenitor cells.

The foregoing compositions, methods and uses are intended to be illustrative and not limiting. Using the teachings provided herein other variations on the compositions, methods and uses will be readily available to one of skill in the art.

### EXAMPLES

The following examples are offered to illustrate, but not to limit the claimed invention.

### Example 1

Identification of envelope proteins that enable more efficient transduction of human CD34+ cells and improved transduction methods of human CD34+ cells by expressing human L-selectin and/or an arenavirus envelope protein.

### Methods

Construction of expression vectors. The pHCMV VSV-G Indiana envelope expression vector was obtained from the Stanford virus core and contains the VSV-G Indiana envelope protein under the control of a human CMV (HCMV) promoter (FIG. 18; SEQ ID NO: 44). An Apa I/Msc I restriction fragment in pHCMV VSV-G Indiana that contains part of the 5′ untranslated region, the coding sequence for the VSV-G Indiana envelope protein, and part of the 3′ untranslated region was replaced by coding regions for other envelope proteins that were synthesized (DNA 2.0, Inc.) and flanked by the same 5′ and 3′ untranslated regions such that only the envelope coding regions were changed. Examples of such plasmids are pHCMV Bas Congo envelope, pHCMV Chandipura envelope, pHCMV Curionopolis envelope, pHCMV Ekpoma-lenvelope, pHCMV Ekpoma-2 envelope, pHCMV Isfahan envelope, pHCMV Kamese envelope, pHCMV Kontonkan envelope, pHCMV Kwatta envelope, pHCMV Le Dantec envelope, pHCMV rabies envelope, pHCMV VSV Alagoas envelope, pHCMV VSV Arizona envelope, pHCMV VSV Carajas envelope, pHCMV VSV Maraba envelope, pHCMV VSV Morreton envelope, pHCMV VSV New Jersey envelope, and pHCMV Machupo envelope.

The human L-selectin expression vector, pCMV6-XL5 human SELL which contains the human L-selectin (gene symbol: SELL) coding region under the control of a CMV promoter was purchased from Origene, Inc (FIG. 19; SEQ ID NO:45). An eGFP lentivirus vector (pCCL MNDU3 eGFP) was obtained from Don Kohn (UCLA) and a map of this vector is is set out as FIG. 20 and also SEQ ID NO: 46. A β-globin lentivirus vector (pCCL GLOBE1 βAS3) was obtained from Fulvio Mavilio (Genethon) (See FIG. 21; SEQ ID NO: 47).

Production of lentiviruses. 293T cells (American Type Culture Collection) were plated in 12.5 ml DMEM media (Invitrogen) with 10% fetal bovine sera (Hyclone) at a density of 1.2×10⁷ cells per 75 cm² flask. Twenty four hours after the cells were plated the media was removed and the cells were washed with 5 ml X-VIVO 15 media with gentamycin (Lonza) and then 12.5 ml of X-VIVO 15 media with 10 mM HEPES was added. For the production of viruses with a single envelope protein, the cells were transfected by mixing 100 μl of OptiMem I media (Invitrogen), 10 μg of lentivirus vector plasmid, 5 μg of pRSV rev (FIG. 22; SEQ ID NO: 48), 5 μg of pMDLg/pRRE (FIG. 23; SEQ ID NO: 49), and 5 μg of the envelope expression plasmid with 100 μg of linear, 25 kDal PEI (VWR), incubating for 10 min at ambient temperature, and then adding that mixture to the cells. For the production of viruses in the presence of L-selectin expression 5 μg of pCMV6-XL5 human SELL (FIG. 19) was added to the above mixture and the amount of envelope expression plasmid was typically reduced from 5 μg to 1 μg. Twenty four hours after the start of transfection the media was removed from the flask, stored at 4° C. and replaced with 12.5 ml of X-VIVO 15 media with gentamycin, 10 mM HEPES. Forty eight hours after the start of transfection the media was removed from the flask and pooled with media from the first harvest. Cells and debris were pelleted from the media by centrifugation at 3,000×g for 15 min at 4° C. The supernatant was filtered through a sterile 0.45 μM pore size filter unit (Steri-flip; Millipore) to remove any remaining 293T cells since some of them may have been transduced during the virus production and could be confused with transduced target cells. The crude virus was treated with Benzonase (Sigma) at a final concentration of 50 U/ml for 30 min at 37° C. to reduce the amount of plasmid remaining from the transfection. The virus was concentrated approximately 100-fold by ultrafiltration using Amicon Ultra-15 units (Millipore, 100 kDal molecular weight cut off) that contain regenerated cellulose membranes to about 0.2 ml. The virus was aliquoted into various single-use sizes and stored at −80° C. until use. All viruses were only thawed once and then used. The virus concentration was determined using a p24 capsid ELISA (Clontech, Inc.). Since the infectivity of viruses containing different envelopes can vary widely on different cells an assay that determines “particle number” (p24 capsid ELISA) rather than the infectious titer on a cultured cell was used to ensure known amount of viral particles were added to transductions.

Transduction of human CD34 cells. Bone-marrow derived human CD34+ cells were purchased from Lonza. Two days prior to transduction untreated 48-well plates (VWR cat#73521-144) were coated with 0.25 ml PBS containing 20 μg/ml retronectin (Lonza) for 24 hrs at 4° C. The PBS/retronectin was removed and the plate was blocked with PBS, 2% bovine serum albumin for at least 30 min at ambient temperature. The CD34+ cells were thawed, pelleted, then resuspended in X-VIVO15 media with gentamycin, 50 ng/ml human c-kit ligand (R&D Systems), 20 ng/ml human IL-3 (R&D Systems), 50 ng/ml human Flt-3 ligand (R&D Systems), and 50 ng/ml human thrombopoietin (R&D Systems), using 0.25 ml per well. The cells were incubated at 37° C. for 24 hrs. and then the desired amount of lentivirus was added for transduction. Additional media was added if necessary to keep the cell density under 1×10⁶ cells/ml.

Determination of % eGFP+ cells in CD34 cells transduced with a lentiviral vector that contains an eGFP expression cassette. Three days after the start of viral transduction cells were collected and pelleted in a 96-well, V-bottom plate for at 20° C. for 5 mins at 300×g. The media was removed and the cells were washed with 200 μl PBS, 1% FBS. The cells were pelleted at 20° C. for 5 minutes at 300×g and the PBS, 1% FBS was removed. The cells were resuspended in 200 μl PBS, 2% paraformaldehyde, 1% FBS. The % eGFP+ cells were determined using an Accuri flow cytometer (BD Biosciences).

Determination of integrated vector copy number of a lentiviral vector that contains a β-globin minigene in transduced human CD34 cells. Three weeks after the start of viral transduction the cells were collected and pelleted in 1.5 ml microfuge tubes at 20° C. for 5 min at 300×g. The media was removed and the cells were resuspended in 200 μl PBS. Genomic DNA was prepared using a DNeasy Blood & Tissue Kit (Qiagen, Inc.) according to the manufacturer's instructions. The genomic DNA was subjected to three quantitative polymerase chain reactions (Q-PCRs) to measure the copy number of the integrated lentivirus, the copy number of a single copy gene (to count the number of cells in the sample), and the amount of plasmid that may remain from the transfection (which can interfere with accurate quantitation of the lentiviral genome since the lentiviral genome is completely contained within the transgene plasmid).

The sequences of the primers and probes used for the Q-PCRs are as follows.

For quantitation of the copy number of the integrated lentivirus the target for Q-PCR was a sequence that overlaps the viral RNA genome packaging sequence (psi).

The sequence of the forward primer used is:
(SEQ ID NO: 50)
5′-ACTTGAAAGCGAAAGGGAAAC-3′
The sequence of the reverse primer used is:
(SEQ ID NO: 51)
5′-CGCACCCATCTCTCTCCTTCT-3′
The sequence of the probe used is:
(SEQ ID NO: 52)
5′-6FAM-AGCTCTCTCGACGCAGGACTCGGC-TAMRA-3′
The DNA used as a standard was:
(SEQ ID NO: 47)
pCCL-GLOBE1-βAS3 .

For quantitation of the copy number of a single copy gene the target for Q-PCR was the human RNAse P gene. TaqMan RNAse P Detection Reagents (Applied Biosystems) consisting of premixed primers and a probe were used for Q-PCR. The DNA used as a standard was human DNA provided with the reagents.

For quantitation of residual plasmid the target for Q-PCR was a sequence in the SV40 origin of replication which is found only in the pCCL-based lentiviral vector backbone outside of the region encoding the viral RNA genome and also is not in any other plasmid used for virus production.

The sequence of the forward primer used is:
(SEQ ID NO: 53)
5′-CTCTGAGCTATTCCAGAAGTAGTG-3′
The sequence of the reverse primer used is:
(SEQ ID NO: X)
5′-CAGTGAGCGCGCGTAATA-3′
The sequence of the probe used is:
(SEQ ID NO: 54)
5′-6FAM-GACGTACCCAATTCGCCCTATAGTG-TAMRA-3′
The DNA used as a standard was:
(SEQ ID NO: 47)
pCCL-GLOBE1-PAS3.

The Taqman Fast advanced master mix, 2× (Applied Biosystems) was used and the Q-PCR was performed on a Roche LightCycler II instrument. The copy number of the lentiviral genome (minus the amount of residual plasmid) divided by the copy number of the single copy gene is the average vector copy number (VCN) per transduced cell. Typically the amount of residual vector plasmid DNA was 1% of the copy number of the lentiviral genome and therefore was insignificant.

Inhibition of human L-selectin-enhanced transduction of human CD34+ cells by a neutralizing antibody to human L-selectin. Two nanograms (measured by p24 capsid ELISA) of VSV-G Indiana pseudotyped lentivirus with a CCL-MNDU3-eGFP genome was produced as above in the presence or absence of human L-selectin. The viruses were incubated with or without 10 μM anti-human L-selectin antibody (clone DREG56; Thermo Fisher) for 30 min at 37° C. in a volume of 20 μl and then added to cytokine-stimulated human CD34+ cells which were in 0.25 ml media, prepared as above. Three days after the start of transduction the % eGFP+ cells was determined as described above.

### Results

Strategy for selection of rhabdovirus envelopes to screen for those that enable transduction of human CD34 cells. Approximately 6000 rhabdovirus envelope sequences were found in GenBank. That number was narrowed to 10 envelope sequences by determining which were isolated from humans or primates or those in which there was serological evidence they can infect a primate, those which had never been assembled with a recombinant lentivirus, and those for which a complete sequence of the coding region was available to construct an envelope protein expression vector. The 11 envelope proteins that met those criteria are from the following rhabdoviruses: VSV Arizona, Bas Congo, Curionopolis, Ekpoma-1, Ekpoma-2, Isfahan, Kamese, Kontonkan, Kwatta, Le Dantec, and rabies. In addition the envelope protein from the Chandipura rhabdovirus, which had been tested by others (Hu, et al., 2016), was also tested. FIG. 1 shows the phylogenetic relationships of these viral envelope proteins, the rhabdovirus subfamilies they belong to, and their % amino acid identity to the VSV Indiana envelope protein.

Representative envelope proteins from most rhabdovirus subfamilies do not enable transduction of human CD34+ cells. Of the rhabdovirus envelope proteins listed above, all of them except Le Dantec were able to transduce 293T cells by a lentivirus with the CCL-MNDU3-eGFP genome (Table 1). This shows that, with the exception of the Le Dantec envelope, all of the expression plasmids encode functional envelope proteins. Transduction of cell lines by Chandipura and rabies has been observed previously. The level of transduction of 293T cells by the Isfahan envelope, which has not been reported previously, suggests it may be useful for transduction of other cultured cell lines.

In contrast, only the VSV Arizona and Indiana envelope proteins enabled efficient transduction of human CD34+ cells by a lentivirus with the CCL MNDU3 eGFP genome (Table 2).

TABLE 1
Transduction of 293T cells with lentiviruses produced using
the indicated envelope protein and an eGFP genome.

| Envelope protein | % eGFP + 293T cells |
|---|---|
| Experiment #1: | |
| VSV (Indiana) | 82.7% |
| Chandipura | 52.6% |
| Isfahan | 28.1% |
| Rabies | 7.3% |
| Kamese | 6.9% |
| Ekpoma-2 | 0.8% |
| Bas Congo | 0.6% |
| Kwatta | 0.4% |
| Ekpoma-1 | 0.1% |
| Curionopolis | 0.01% |
| Kotonkan | 0.01% |
| Le Dantec | 0.0005% |
| No envelope | 0.0005% |
| Experiment #2: | |
| VSV (Indiana) | 100% |
| VSV (Arizona) | 100% |
| No envelope | <0.1% |

Lentivirus was produced using a pCCL MNDU3 eGFP genome and the indicated envelope protein. 293T cells were transduced using 1 ng p24 per well (on a 24 well plate). % GFP+ cells was determined 1 day post-infection.

TABLE 2
Transduction of human CD34+ cells with lentiviruses produced
using indicated the envelope protein and an eGFP genome.

| Envelope protein | % eGFP + CD34 cells |
|---|---|
| Experiment #1: | |
| VSV (Indiana) | 43% |
| Bas Congo | 1% |
| Rabies | 1% |
| Curionopolis | <0.1% |
| Ekpoma-1 | <0.1% |
| Ekpoma-2 | <0.1% |
| Isfahan | <0.1% |
| Kamese | <0.1% |
| Kotonkan | <0.1% |
| Kwatta | <0.1% |
| Chandipura | <0.1% |
| Le Dantec | <0.1% |
| No envelope | <0.1% |
| Experiment #2: | |
| VSV (Indiana) | 75% |
| VSV (Arizona) | 81% |
| No envelope | <0.1% |

Lentivirus was produced using a pCCL-MNDU3-eGFP genome and the indicated envelope protein. Cytokine-stimulated human CD34+ cells were transduced using 10 ng p24 per well (on a 48 well plate). % GFP+ cells was determined 3 days post-infection.

All new world-derived vesiculovirus envelope proteins transduce human CD34+ cells. Since another vesiculovirus envelope protein (VSV-G Arizona) besides VSV-G Indiana transduced human CD34+ cells, representatives of all known vesiculoviruses for which complete coding regions were available were tested for transduction of human CD34+ cells regardless of whether there was evidence that they could infect humans. The 5 additional VSV envelope proteins that were tested were from the following VSV strains: Alagoas, Carajas, Maraba, Morreton, and New Jersey. These envelope proteins vary widely in their amino acids sequence identity to the VSV Indiana envelope sequences (Table 3). All of these envelope proteins enabled transduction of human CD34+ cells (FIG. 2) although with different efficiencies. Lentiviruses with the Alagoas, New Jersey, and Carajas envelope proteins transduced human CD34+ cells less efficiently than those with a VSV Indiana envelope while lentiviruses with the Morreton, Arizona, and Maraba envelope proteins transduced human CD34+ cells more efficiently than those with a VSV Indiana envelope.

TABLE 3
% amino acid identity of representative vesiculovirus envelope
proteins to the VSV-G (Indiana) envelope protein.

| | % identity to VSV-G_ |
|---|---|
| Envelope protein | (Indiana) envelope protein |
| VSV (Indiana) | 100% |
| VSV (Morreton) | 85% |
| VSV (Maraba) | 78% |
| VSV (Alagoas) | 63% |
| VSV (Carajas) | 55% |
| VSV (New Jersey) | 50% |
| VSV (Arizona) | 50% |

Between these results and those published in the literature, there are 3 vesiculovirus envelopes that poorly mediate transduction of human CD34+(Isfahan, Piry, Chandipura) cells and 8 that can significantly transduce (VSV (Arizona), VSV (Indiana), VSV (New Jersey), Morreton, Maraba, Alagoas, Carajas, Cocal). The 3 vesiculovirus envelopes that poorly mediate transduction of human CD34+ cells are derived from old world vesiculoviruses, while the 8 that can significantly mediate transduction of human CD34+ cells are derived from new world vesiculoviruses (FIG. 3).

By comparing the amino acid sequences of various vesiculovirus envelope proteins, there are 31 amino acids found in all envelopes that significantly mediate transduction of human CD34+ cells but none of those amino acids at those positions are found in envelopes that poorly transduce human CD34+ cells (FIG. 4). This set of 31 amino acids is a “CD34+ cell transduction determinant” and may be useful in predicting whether any vesiculovirus envelopes discovered in the future are capable of human CD34+ cell transduction. Furthermore those amino acids could be engineered into vesiculovirus envelopes that poorly transduce human CD34 cells in order to potentially convert them into an envelope protein that can mediate transduction of human CD34 cells. The correlation of phylogeny and function may be due to binding of different receptors by old world and new world vesiculoviruses. The Cocal and VSV Indiana envelope proteins are known to bind to LDL-R to enter cells. Transduction by the VSV Arizona envelope was inhibited by soluble LDL-R (data not shown) suggesting it may also bind to LDL receptor. Therefore, it may be the case that all of the new world vesiculoviruses bind to LDL-R to enter cells while the old world vesiculoviruses may bind to a different (currently unidentified) receptor. Most of the 31 amino acids comprising the CD34+ cell transduction determinant are buried in the pre-fusion structure of VSV-G Indiana. The most surface exposed amino acids in the CD34+ cell transduction determinant in the pre-fusion structure of VSV-G Indiana are Asp 290, Val 291, Glu 292, Ser 305, and Gly 365 (FIG. 5).

Examination of the phylogenetic relationships between New World vesiculovirus envelope proteins suggested that they group into separate branches and furthermore those branches may correlate to the efficiency of human CD34+ cell transduction. Envelopes on branches that contain the Alagoas and Carajas envelopes were less efficient at transduction of human CD34+ cells than those on a different branch (Maraba, Morreton, Indiana, and Cocal). Therefore, there may also be a “CD34+ cell transduction efficiency determinant sequence.”

### Example 2—Enhanced Transduction Efficiency with L-Selectin

In order to improve transduction of CD34+ cells further, non-envelope protein ligands that might be assembled on the surface of a lentivirus and can bind to the surface of CD34+ cells were screened. CD34 is expressed on CD34+ cells and L-selectin is a known ligand that binds CD34. Production of lentiviruses in the presence of L-selectin expression resulted in a lentivirus with improved CD34+ cell transduction (FIG. 6). The magnitude of the effect depended on the desired VCN in the transduced cells. For example, to achieve a VCN of one, 5-fold less virus could be used when virus was produced in the presence of L-selectin compared to without L-selectin (FIG. 8). To achieve a VCN of two, in this particular example, 8-fold less virus could be used. L-selectin expression in 293T producer cells (for example using 1 μg of the human L-selectin expression vector pCMV6-XL5 huSELL and 5 μg of the VSV-G Indiana envelope expression vector pHCMV) typically reduced virus production from 1.0-1.5 fold (data not shown). Therefore, even with a slight reduction in production, there would still be a net gain in transduction.

CD52 is also expressed on CD34+ cells and SIGLEC10 is a known ligand for CD52. Production of lentiviruses in the presence of SIGLEC10 expression did not result in a lentivirus with improved CD34+ cell transduction (FIG. 7), compared to lentivirus produced in the presence of L-selectin. Furthermore SIGLEC10 expression in 293T producer cells dramatically reduced virus production (data not shown). Thus SIGLEC10 ligand co-expression during lentivirus production does not appear to enhance CD34+ transduction.

An optimized condition for producing virus in the presence of L-selectin (1 μg VSV-G Indiana plasmid, 5 μg L-selectin plasmid) was compared to an optimized virus production method that is commonly used (with 5 μg VSV-G Indiana plasmid) and one in which the amount of VSV-G Indiana plasmid was reduced to that in the optimized production containing L-selectin (1 μg VSV-G Indiana plasmid). As expected there was a slight reduction in efficiency of human CD34+ cell transduction when the amount of the VSV-G Indiana plasmid was reduced from 5 μg to 1 μg (FIG. 8). However adding 5 μg of the L-selectin expression vector more than compensated for that slight reduction. In this experiment the VSV-G Indiana-enveloped virus produced in the presence of L-selectin was about 5-fold more efficient than the VSV-G Indiana-enveloped virus.

The effect of adding 5 μg of the L-selectin expression vector to 5 μg of the VSV-G Indiana expression vector was also assessed. Adding 5 μg of the L-selectin expression vector to 5 μg of the VSV-G Indiana expression vector did not increase lentivirus transduction efficiency (FIG. 9). Furthermore the combination of 5 μg of the L-selectin expression vector and 5 μg of the VSV-G Indiana expression vector reduced virus production 3-fold. In this experiment, the VSV-G Indiana-enveloped virus produced in the presence of L-selectin was about 6-fold more efficient than the VSV-G Indiana-enveloped virus. Since reduction of the amount of pHMCV VSV-G Indiana plasmids used to produce the virus from 5 μg to 1 μg in the presence of 5 μg of the human L-selectin plasmid did not reduce the enhancement of CD34+ cell transduction but did increase lentivirus production it is possible lentivirus production could be increased further by reducing the amount of envelope expression vector more. Virus produced in the absence of any envelope protein produced at least 2-3 fold more viral particles than when 5 μg of VSV-G envelope expression plasmid (per 75 cm² flask) is used to produce virus. If the amount of envelope expression plasmid can be reduced to a completely nontoxic level similar to what is observed when no envelope expression plasmid is used, but enhanced transduction can be maintained by including a human L-selectin expression vector in the virus production then not only could there be enhanced transduction of CD34+ cells but also enhanced viral production.

L-selectin also enhanced the transduction efficiency of the Maraba (Table 4), Morreton (FIG. 14) and Carajas (FIG. 15) vesiculovirus envelope proteins. In initial experiments, the enhancement of Maraba envelope-mediated transduction of human CD34+ cells was typically 3 to 6 fold and in one experiment there was a 10-fold enhancement of Morreton envelope-mediated transduction of human CD34+ cells.

TABLE 4
Expression of human L-selectin in virus
producer cells enhances transduction of human CD34+
cells by the Maraba vesiculovirus envelope protein.

| Envelope | ng viral capsid needed to obtain VCN = 0.5 |
|---|---|
| Indiana | 54.6 |
| Maraba | 10.0 |
| Maraba + L-selectin | 3.3 |

### Example 3—L-Selectin can be Incorporated into Lentiviruses

L-selectin expression in lentivirus producing cells could enhance transduction of CD34+ cells by such lentiviruses by mechanisms that may or may not involve incorporation of L-selectin into the virus. The simplest hypothesis for why L-selectin expression in lentivirus producing cells resulted in lentiviruses exhibiting enhanced transduction of CD34+ cells is that L-selectin is being incorporated into the virus and that this incorporation resulted in improved binding to CD34+ cells. Binding of viruses to cells is well known to be a rate limiting step of transduction. Alternatively, L-selectin expression in lentivirus producing cells could indirectly affect infectivity of lentiviruses by, for example, reducing degradation of VSV-G or enhancing incorporation of VSV-G into the lentivirus. The amount of VSV-G or other envelope on a virus is known to correlate to its transduction efficiency.

To determine if L-selectin might be incorporated into a lentivirus, lentiviruses with a CCL-MNDU3-eGFP genome were produced in the presence or absence of human L-selectin and then each virus was incubated with or without 10 μM of a neutralizing antibody to human L-selectin. Those samples were then used to transduce cytokine-stimulated CD34+ cells and % eGFP+ cells were measured 3 days after the start of transduction. The results are shown in FIG. 10. First, production of lentivirus in the presence human L-selectin enhanced transduction about 2-fold from 16.2% eGFP+ cells to 27.1% eGFP+ cells. Second addition of the L-selectin neutralizing antibody to virus produced in the absence of human L-selectin did not significantly reduce its ability to transduce CD34+ cells (15.6% eGFP+ cells). However addition of the L-selectin neutralizing antibody to virus produced in the presence of human L-selectin reduced the L-selectin enhanced amount of CD34+ transduction (=27.1%-16.2%=10.9%) by 79% from 27.1% eGFP+ cells to 18.5% eGFP+ cells (27.1%-18.5%=8.6%; 8.6%/10.9% is a 79% reduction). This indicates that human L-selectin can be incorporated into lentivirus particles when it is expressed in virus producing cells and that it plays an important role in contributing to the enhanced transduction of CD34+ cells by such lentiviruses.

### Example 4—Transduction of Cells that do not Express CD34 is not Enhanced when a Lentivirus is Produced in Cells Expressing L-Selectin

To provide more evidence L-selectin was incorporated into lentiviruses, cells that do not express CD34 (293T cells) were transduced with virus (CCL-MNDU3-eGFP genome) produced in the presence or absence of human L-selectin. If human L-selectin is being incorporated into lentiviruses and improving the binding of lentiviruses to cells, then virus produced in the presence of human L-selectin should not transduce such CD34-cells better compared to virus produced in the absence of human L-selectin. As shown in FIG. 11, virus produced in the presence of human L-selectin did not transduce 293T cells (cells that are re CD34-) better than virus produced in the absence of human L-selectin. If L-selectin expression was causing increased infectivity by indirect means such as increasing the amount of VSV envelope in the virus then such viruses should have increased infectivity on a variety of cells susceptible to transduction by VSV enveloped lentiviruses.

### Example 5—L-Selectin Protein Co-Expression During Lentivirus Production Improves Transduction by Lentivirus Vector Pseudotyped by Many Different Vesiculovirus Envelope Proteins

Since co-expression of L-selectin during lentivirus production improved VSV-G (Indiana)-mediated transduction of human CD34+ cells, we decided to examine this transduction enhancement effect of L-selectin co-expression occurred with other vesculovirus envelope proteins during vector production. There is particular interest in the Maraba envelope since lentivirus pseudotyped with Maraba envelope exhibits improved transduction of CD34+ cells compared to VSV-G Indiana envelope (FIG. 12). The dose-relationship between Maraba envelope and L-selectin was examined by altering the amount of Maraba envelope expression plasmid (from 1 μg to 0.25 μg) against L-selectin expression plasmid (from 5 μg to 1 μg) for transfection (with lentiviral helper plasmids and pCCL-GLOBE1-bAS3) into a T-75 flask of 293T producer cells. Lentivirus from each production condition was processed as described above and used to transduce human CD34+ cells at 1, 3, 10, 30 ng of p24gag (FIG. 12). Interestingly, as decreasing amounts of Maraba envelope plasmid was transfected into the 293T cells, the resulting Maraba pseudotyped lentivirus showed improved transduction of CD34+ cells as measured by VCN analysis. Accordingly, decreasing the amount of L-selectin co-expression plasmid used to transfect vector producing 293T cells, also improved the transduction efficiency of the resulting Maraba pseudotyped lentivirus (compare VCN transduction using lentiviruses produced using 0.25 μg Maraba plasmid with 5 μg or 1 μg L-selectin plasmid during co-transfected of 293T cells—FIG. 12, bottom graph) The VCN transduction results indicate that the optimal range for Maraba envelope expression plasmid during lentivirus production is between 0.25 μg to 0.5 μg, while the optimal range of L-selectin expression plasmid is between 1 μg to 2.5 μg under the transfection conditions described herein. The ratio of vesiculovirus envelope:L-selectin expression plasmids transfected during vector production should be within the range of 1:2 to 1:5 to achieve the maximum transduction enhancement effects. The use of other heterologous viral envelope proteins to pseudotype lentiviruses might require different envelope: L-selectin plasmid ratios for virus production for enhancement of viral transduction.

In order to show that Maraba envelope plus L-selectin-mediated lentivirus transduction is robust, single preparations of lentiviruses pseudotyped with VSV-G Indiana envelope (prototypical control) or with Maraba envelope plus L-selectin was used to transduce CD34+ cells from three separate donors (FIG. 13). VCN analysis of the CD34+ cell genomic DNA showed that there was at least a two-fold increase in VCN when lentivirus was pseudotyped with Maraba envelope and L-selectin, compared to the control VSV-G pseudotyped lentivirus.

L-selectin co-expression during vector production also improved the transduction of other vesiculovirus envelopes such as Morreton (FIG. 14) and Carajas (FIG. 15). Lentivirus pseudotyped with Carajas envelope, with or without L-selectin co-expression during vector production, was compared to VSV-G Indiana envelope or Alagoas envelope pseudotyped lentiviruses (FIG. 15). Similar to FIG. 2, Carajas envelope lentivirus transduces CD34+ cells to a similar extent as to lentivirus pseudotyped with VSV-G Indiana, while Alagoas envelope lentivirus mediates lower levels of CD34+ transduction compared to VSV-G Indiana lentivirus. Significantly there is enhanced CD34+ transduction when L-selectin is co-expressed with Carajas envelope during lentivirus production. We did not observe any enhancement in Alagoas envelope mediated CD34+ transduction if L-selectin is expressed during lentivirus production; however it is possible that the levels of Alagoas envelope expression or the ratio of Alagoas envelope and L-selectin expression during lentivirus production is not optimal for this particular vesiculovirus envelope.

In summary, these results demonstrate that (1) Novel vesiculovirus envelopes (including Maraba, Morreton, VSV-G Arizona, and Carajas envelopes) are able to pseudotype and mediate efficient lentivirus transduction of primary human CD34+ cells to the same level or greater than the prototypical VSV-G Indiana vesiculovirus envelope. (2) Co-expression of human L-selectin protein in the lentivirus producer cells generates VSV-G pseudotyped lentivirus with improved CD34+ cell transduction properties. (3) L-selectin co-expression in lentivirus producer cells can improve CD34+ cell transduction of lentiviruses pseudotyped with many different vesiculovirus envelope proteins (including Maraba, Morreton and Carajas envelopes). (4) The ratio of vesiculovirus envelope to L-selectin expression plasmids in the lentivirus producer cells may be an important factor in the magnitude of the lentivirus transduction enhancement of human primary CD34+ cells. (5) The transduction improvements describes above have been shown to work with both GFP-reporter lentiviruses and with human beta-globin expression lentiviruses, and thus are applicable to lentiviruses used for experimentation or to treat hemaglobinopathies or other clinical conditions.

### Example 6—Transduction of Human CD34+ Cells by Lentiviruses Pseudotyped with the Machupo Arenavirus Envelope Protein

Besides CD34, another cell surface protein that is expressed on CD34+ cells is the transferrin type 1 receptor (CD71), which is expressed on most mammalian cells. The Machupo arenavirus is a human pathogen and utilizes the human transferrin type 1 receptor to infect human cells. Transferrin (a common component of cell culture media) does not inhibit infection of cells by Machupo virus (Radoshitzky, S. R., et al., 2007). Furthermore a crystal structure of the Machupo GP1 envelope protein (Carvallo strain) bound to the human transferrin type 1 receptor has been determined (Abraham J., et al. (2010). Besides potentially being useful for envelope protein engineering it can be seen from the Machupo GP1 envelope protein-human transferrin type 1 receptor structure that the Machupo envelope binds to the human transferrin type 1 receptor in a region that would not conflict with the binding of transferrin to the human transferrin type 1 receptor which supports the cell culture experiment reported by Radoshitzky. These properties made it compelling to test the ability of the Machupo virus envelope protein to pseudotype lentivirus and mediate transduction of human CD34+ cells Lentiviruses with a CCL GLOBE1 βAS3 genome were produced with the VSV-G Indiana envelope (positive control; 5 μg per 75 cm² flask), and the Machupo envelope (Carvallo strain) using 2 different amounts of expression plasmid (1 or 5 μg per 75 cm² flask). In addition, the lentivirus produced with 1 μg per 75 cm² flask of the expression plasmid for the Machupo envelope (Carvello strain) was also produced in the presence human L-selectin expression (1 μg envelope expression plasmid and 5 μg human L-selectin expression plasmid per 75 cm² flask). The Machupo virus envelope was capable of mediating transduction of human CD34+ cells about as efficiently as VSV-G Indiana and co-expression of L-selectin (SELL) in the virus producer cells enhanced transduction (FIG. 16).

Arenavirus envelope proteins can be grouped phylogenetically into either old world or new world-derived isolates (FIG. 17). This in turn correlates with their tropism and receptor usage. Old world-derived arenavirus envelope proteins typically utilize α-dystroglycan to infect cells while new world-derived arenavirus envelope proteins may utilize the transferrin type 1 receptor to infect human cells and appear to have common sequences that determine receptor binding (Radoshitzky, et al., 2011). Two old world-derived arenavirus envelope proteins (from LCMV and Lassa virus) have previously been tested for CD34+ cell transduction and found to transduce human CD34+ cells very poorly (Sandrin, et al., 2002). However a lentivirus pseudotyped with a new world-derived arenavirus envelope protein (Machupo) can transduce human CD34 cells well (FIG. 16). Therefore, as was the case with vesiculovirus envelopes, other new world-derived arenavirus envelope proteins may transduce CD34+ cells more or less efficiently than the Machupo virus envelope and would be worth testing. There appears to be various phylogenetic subdivisions within the new world-derived arenavirus envelope proteins (FIG. 17) and these different subgroups may transduce CD34+ cells more or less efficiently than other subgroups. For example the Machupo, Junin, Ocozocoautla, and Tacaribe envelope proteins may constitute one Glade that can mediate transduction of human CD34+ cells, but with different efficiencies.

### REFERENCES

Abraham J., et al (2010) Structural basis for receptor recognition by New World hemorrhagic fever arenaviruses. Nat Struct Mol Biol. 17:438-44.
Amirache, F., et al. (2014) Mystery solved: VSV-G-LVs do not allow efficient gene transfer into unstimulated T cells, B cells, and HSCs because they lack the LDL receptor. Blood. 123:1422-4.
Bandala-Sanchez, E., et al. (2013) T cell regulation mediated by interaction of soluble CD52 with the inhibitory receptor Siglec-10. Nat Immunol. 7:741-8.

Booth C., et al. (2016) Treating Immunodeficiency through HSC Gene Therapy. Trends Mol Med. 22:317-27.
Brendel, et al. (2015) CD133-targeted gene transfer into long-term repopulating hematopoietic stem cells. Mol Ther. 23:63-70.
Farinelli G., et al. (2014) Lentiviral vectors for the treatment of primary immunodeficiencies. J Inherit Metab Dis. 37:525-33.
Finkelshtein D., et al (2013) LDL receptor and its family members serve as the cellular receptors for vesicular stomatitis virus. Proc Natl Acad Sci USA. 110:7306-11.
Hu, S. (20160 Pseudotyping of lentiviral vector with novel vesiculovirus envelope glycoproteins derived from Chandipura and Piry viruses. Virology. 15:162-8.
Humbert, O., et al. (2016) Development of Third-generation Cocal Envelope Producer Cell Lines for Robust Lentiviral Gene Transfer into Hematopoietic Stem Cells and T-cells. Mol Ther. 24:1237-46.
Klabusay, M., et al (2007) Different levels of CD52 antigen expression evaluated by quantitative fluorescence cytometry are detected on B-lymphocytes, CD 34+ cells and tumor cells of patients with chronic B-cell lymphoproliferative diseases. Cytometry B Clin Cytom. 5:363-70.
Negre O., et al. (2016) Gene Therapy of the β-Hemoglobinopathies by Lentiviral Transfer of the β(A(T87Q))-Globin Gene. Hum Gene Ther. 27:148-65.
Nielsen, J. S., et al. (2009) CD34 is a key regulator of hematopoietic stem cell trafficking to bone marrow and mast cell progenitor trafficking in the periphery. Microcirculation. 6:487-96.
Radoshitzky, S. R., et al. (2007). Transferrin receptor 1 is a cellular receptor for New World haemorrhagic fever arenaviruses. Nature. 446:92-6.
Radoshitzky S. R., et al. (2011) Machupo virus glycoprotein determinants for human transferrin receptor 1 binding and cell entry. PLoS One. 6:e21398.
Rastall D. P., et al. (2015) Recent advances in gene therapy for lysosomal storage disorders. Appl Clin Genet. 24:157-69.
Sandrin, V., et al., (2002). Lentiviral vectors pseudotyped with a modified RD114 envelope glycoprotein show increased stability in sera and augmented transduction of primary lymphocytes and CD34+ cells derived from human and nonhuman primates. Blood. 100:823-32.

1. A recombinant lentivirus capable of transducing a hematopoietic stem cell, said recombinant lentivirus comprising i) a heterologous transgene, ii) a viral envelope protein, and iii) a protein that is a ligand for binding to CD34+ cells.

2. The recombinant lentivirus of claim 1, wherein the viral envelope protein is a vesiculovirus envelope protein.

3. The recombinant lentivirus of claim 2, wherein the vesiculovirus envelope protein originates from a species of vesiculovirus selected from the group consisting of Vesicular Stomatitis Virus G (VSV-G), Morreton, Maraba, Cocal, Alagoa and Carajas.

4. The recombinant lentivirus of claim 1, wherein the viral envelope protein is an arenavirus envelope protein.

5. The recombinant lentivirus of claim 4, wherein the arenavirus envelope protein originates from a Machupo virus.

6. The recombinant lentivirus of any one of claims 1-3, wherein the viral envelope protein comprises an amino acid sequence having at least 70%, 75%, 80%, 85%, 90%, 95%, 96%, 97%, 98% or 99% sequence identity to the amino acid sequence of SEQ ID NO:2, 4, 6, 8, 10, 12, 14, 16, 18, 20, 22, 24, 26, 28, 30, 32, 34, 36, or 43 when the sequence comparison is carried out over the entire length of the two sequences.

7. The recombinant lentivirus of any one of claims 1-3, wherein the viral envelope protein comprises an amino acid sequence of SEQ ID NO:2, 4, 6, 8, 10, 12, 14, 16, 18, 20, 22, 24, 26, 28, 30, 32, 34, 36, or 43.

8. The recombinant lentivirus of any one of claims 1-3, wherein viral envelope protein consists essentially of the amino acid sequence of SEQ ID NO:2, 4, 6, 8, 10, 12, 14, 16, 18, 20, 22, 24, 26, 28, 30, 32, 34, 36, or 43.

9. The recombinant lentivirus of any one of claims 1-3, wherein the viral envelope protein consists of the amino acid sequence of SEQ ID NO:2, 4, 6, 8, 10, 12, 14, 16, 18, 20, 22, 24, 26, 28, 30, 32, 34, 36, or 43.

10. The recombinant lentivirus of any one of claim 1-3 or 6-9 wherein said viral envelope protein comprises at least one of the 31 amino acids within the CD34 cell transduction determinant shown in FIG. 4 at its respective location.

11. The recombinant lentivirus of any one of claim 1-3 or 6-9, wherein said viral envelope protein comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or all 31 of the 31 amino acids within the CD34 cell transduction determinant shown in FIG. 4 at their respective locations.

12. The recombinant lentivirus of claim 4 or 5, wherein the viral envelope protein comprises an amino acid sequence having at least 70%, 75%, 80%, 85%, 90%, 95%, 96%, 97%, 98% or 99% sequence identity to the amino acid sequence of SEQ ID NO:41, when the sequence comparison is carried out over the entire length of the two sequences.

13. The recombinant lentivirus of claim 4 or 5, wherein the viral envelope protein comprises an amino acid sequence of SEQ ID NO:41.

14. The recombinant lentivirus of any one of claims 1-13, wherein the hematopoietic stem cell is a human cell.

15. The recombinant lentivirus of any one of claims 1-13, wherein the hematopoietic stem cell is a human CD34+ cell.

16. The recombinant lentivirus of any one of claims 1-15, wherein said recombinant lentivirus further comprises a vector; and wherein the vector comprises said heterologous transgene operably linked to a promoter.

17. The recombinant lentivirus of any one of claims 1-16, wherein said recombinant lentivirus comprises a self-activating (SIN) LTR.

18. The recombinant lentivirus of any one of claims 1-17, wherein the heterologous transgene encodes a human protein.

19. The recombinant lentivirus of any one of claims 1-18, wherein the heterologous transgene encodes a human hemoglobin protein.

20. The recombinant lentivirus of any one of claims 1-19, wherein the protein that is a ligand for binding to human CD34+ cells is present on the surface of said recombinant lentivirus.

21. The recombinant lentivirus of any one of claims 1-20, wherein the protein that is a ligand for binding to human CD34+ cells is L-selectin.

22. The recombinant lentivirus of any one of claims 1-21, wherein the protein that is a ligand for binding to human CD34+ cells comprises an amino acid sequence having at least 70%, 75%, 80%, 85%, 90%, 95%, 96%, 97%, 98% or 99% sequence identity to the amino acid sequence of SEQ ID NO:39, when the sequence comparison is carried out over the entire length of the two sequences.

23. The recombinant lentivirus of any one of claims 1-21 wherein the protein that is a ligand for binding to human CD34+ cells comprises the amino acid sequence of SEQ ID NO:39.

24. The recombinant lentivirus of any one of claims 1-21, wherein the protein that is a ligand for binding to human CD34+ cells consists essentially of the amino acid sequence of SEQ ID NO:39.

25. The recombinant lentivirus of any one of claims 1-21, wherein the protein that is a ligand for binding to human CD34+ cells consists of the amino acid sequence of SEQ ID NO:39.

26. The recombinant lentivirus of any one of claims 1-25, wherein the recombinant lentivirus is produced by a cell having a concentration ratio of vector expressing the envelope protein and the vector expressing L-selectin ranging from 1:2 to 1:5.

27. The recombinant lentivirus of any one of claims 1-25, wherein the concentration ratio of the envelope protein and L-selectin ranges from 1:2 to 1:5.

28. A method of introducing a heterologous transgene into a hematopoietic stem cell comprising the step of transducing said stem cell with a recombinant lentivirus that comprises (i) said heterologous transgene, (ii) a viral envelope protein, and (iii) a protein that is a ligand for binding to CD34+ cells.

29. The method of claim 28, wherein the viral envelope protein is a vesiculovirus envelope protein.

30. The method of claim 29, wherein the vesiculovirus envelope protein originates from a species of vesiculovirus selected from the group consisting of Vesicular Stomatitis Virus G (VSV-G), Morreton, Maraba, Cocal, Alagoa and Carajas.

31. The method of claim 28, wherein the viral envelope protein is an arenavirus envelope protein.

32. The method of claim 31, wherein the arenavirus envelope protein originates from a Machupo virus.

33. The method of claim any one of claims 28-30, wherein the viral envelope protein comprises an amino acid sequence having at least 70%, 75%, 80%, 85%, 90%, 95%, 96%, 97%, 98% or 99% sequence identity to the amino acid sequence of SEQ ID NO:2, 4, 6, 8, 10, 12, 14, 16, 18, 20, 22, 24, 26, 28, 30, 32, 34, 36 or 43, when the sequence comparison is carried out over the entire length of the two sequences.

34. The method of any one of claims 28-30, wherein the viral envelope protein comprises an amino acid sequence of SEQ ID NO:2, 4, 6, 8, 10, 12, 14, 16, 18, 20, 22, 24, 26, 28, 30, 32, 34, 36, or 43.

35. The method of any one of claims 28-30, wherein the amino acid sequence of said viral envelope 5protein consists essentially of the amino acid sequence of SEQ ID NO:2, 4, 6, 8, 10, 12, 14, 16, 18, 20, 22, 24, 26, 28, 30, 32, 34, 36, or 43.

36. The method of any one of claims 28-30, wherein the amino acid sequence of said viral envelope protein consists of the amino acid sequence of SEQ ID NO:2, 4, 6, 8, 10, 12, 14, 16, 18, 20, 22, 24, 26, 28, 30, 32, 34, 36, or 43.

37. The method of any one of claim 28-30 or 33-36, wherein said viral envelope protein comprises at least one of the 31 amino acids within the CD34 cell transduction determinant shown in FIG. 4 at its respective location.

38. The method of any one of claim 28-30 or 33-36, wherein said viral envelope protein comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or all 31 of the 31 amino acids within the CD34 cell transduction determinant shown in FIG. 4 at their respective locations.

39. The method of claims 31 or 32, wherein the viral envelope protein comprises an amino acid sequence having at least 70%, 75%, 80%, 85%, 90%, 95%, 96%, 97%, 98% or 99% sequence identity to the amino acid sequence of SEQ ID NO:41, when the sequence comparison is carried out over the entire length of the two sequences.

40. The method of claim 31 or 32, wherein the viral envelope protein comprises an amino acid sequence of SEQ ID NO:41.

41. The method of any one of claims 28-40, wherein the hematopoietic stem cell is a human cell.

42. The method of any one of claims 28-41, wherein the hematopoietic stem cell is a human CD34+ cell.

43. The method of any one of claims 28-42, wherein said recombinant lentivirus comprises a vector; wherein the vector comprises said heterologous transgene operably linked to a promoter.

44. The method of any one of claims 28-43, wherein said recombinant lentivirus comprises a self-activating (SIN) LTR.

45. The method of any one of claims 28-44, wherein the heterologous transgene encodes a human hemoglobin protein.

46. The method of any one of claims 28-45, wherein the protein that is a ligand for binding to human CD34+ cells is present on the surface of said recombinant lentivirus.

47. The method of any one of claims 28-46, wherein the protein that is a ligand for binding to human CD34+ cells is L-selectin.

48. The method of any one of claims 28-47, wherein the protein that is a ligand for binding to human CD34+ cells comprises an amino acid sequence having at least 70%, 75%, 80%, 85%, 90%, 95%, 96%, 97%, 98% or 99% sequence identity to the amino acid sequence of SEQ ID NO:39, when the sequence comparison is carried out over the entire length of the two sequences.

49. The method of any one of claims 28-47, wherein the protein that is as a ligand for binding to human CD34+ cells comprises the amino acid sequence of SEQ ID NO:39.

50. The method of any one of claims 28-47, wherein the protein that is as a ligand for binding to human CD34+ cells consists essentially of the amino acid sequence of SEQ ID NO:39.

51. The method of any one of claims 28-47, wherein the protein that is as a ligand for binding to human CD34+ cells consists of the amino acid sequence of SEQ ID NO:39.

52. The method of any one of claims 28-51, wherein the recombinant lentivirus is produced by a cell having a concentration ratio of vector expressing the envelope protein and the vector expressing L-selectin ranging 1:2 to 1:5.

53. The method of any one of claims 28-52, wherein the concentration ratio of the envelope protein and L-selectin ranging 1:2 to 1:5.

54. The method of any one of claims 28-53, wherein said step of transduction is performed on adherent hematopoietic stem cells.

55. The method of any one of claims 28-53, wherein said step of transduction is performed on hematopoietic stem cells in suspension.

56. A recombinant lentivirus capable of transducing a hematopoietic stem cell, said recombinant lentivirus comprising a heterologous transgene and a viral envelope protein that originates from a species of vesiculovirus selected from the group consisting of Vesicular Stomatitis Virus G (VSV-G), Morreton, Maraba, Cocal, Alagoa and Carajas.

57. A recombinant lentivirus capable of transducing a hematopoietic stem cell, said recombinant lentivirus comprising a heterologous transgene and a viral envelope protein comprising at least one of the 31 amino acids within the CD34 cell transduction determinant shown in FIG. 4 at its respective location.

58. A recombinant lentivirus capable of transducing a hematopoietic stem cell, said recombinant lentivirus comprising a heterologous transgene and a viral envelope protein that originates from a species of arenavirus capable of using transferrin receptor type 1 (TfnR1) to infect cells.

59. The recombinant lentivirus of claim 58, wherein the arenavirus envelope protein originates from a Machupo virus.

60. A composition comprising the recombinant lentivirus of any one of claim 1-27 or 56-59 and a pharmaceutically acceptable carrier.

61. A method of treating a hemoglobinopathic condition comprising administering a hematopoietic stem cell transduced with a recombinant lentivirus of any one of claim 1-27 or 56-59 or a composition of claim 60.

62. The method of claim 61 wherein the hemoglobinopathic condition is sickle cell anemia or thalassemias.

63. Use of a hematopoietic stem cell transduced with a recombinant lentivirus of any one of claim 1-27 or 56-59 or a composition of claim 60 for the preparation of a medicament for the treatment of a hemoglobinopathic condition.

64. The use of claim 63 wherein the hemoglobinopathic condition is sickle cell anemia or thalassemias.

66. A composition comprising a hematopoietic stem cell transduced with a recombinant lentivirus of any one of claim 1-27 or 56-59 for treating a hemoglobinopathic condition.

67. The composition of claim 66 wherein the hemoglobinopathic condition is sickle cell anemia or thalassemias.
